(19) United States
(12) Patent Application Publication
(10) Pub. No.: US 2023/0144904 A1
(43) Pub. Date: May 11, 2023

(54) NANOPARTICLE ASSEMBLY FOR CATALYTIC HYDROGEN SENSING

(71) Applicant: Wayne State University, Detroit, MI (US)

(72) Inventors: Long Luo, Detroit, MI (US); Xin Geng, Detroit, MI (US)

(73) Assignee: Wayne State University, Detroit, MI (US)

(21) Appl. No.: 18/055,269

(22) Filed: Nov. 14, 2022

Related U.S. Application Data
(60) Provisional application No. 63/278,451, filed on Nov. 11, 2021.

(51) Int. Cl. C25B 15/023; B82Y 30/00; B82Y 40/00; C25B 11/042

(52) U.S. Cl. CPC C25B 15/023; B82Y 30/00; B82Y 40/00; C25B 11/042

(57) ABSTRACT

Nanoparticle assemblies can be formed and deposited on temperature sensors to create low cost, high sensitivity hydrogen sensors. The nanoparticle assemblies can be formed from nanoparticles within an electrolysis reactor to form grain-boundary connections between the nanoparticles of the nanoparticle assembly. The nanoparticle assembly can be deposited on temperature sensors to create hydrogen gas sensors.

### CROSS-REFERENCE TO RELATED APPLICATION

This US application claims priority to U.S. Provisional Patent Application No. 63/278,451 filed Nov. 11, 2021, which is incorporated by reference in its entirety as if fully set forth herein.

### FIELD OF THE DISCLOSURE

The present disclosure relates to systems and methods for detecting hydrogen in an ambient atmosphere. More specifically, the present disclosure relates to systems and methods for forming nanoparticle assemblies that are capable of detecting hydrogen concentrations approaching the lower flammability concentration.

### BACKGROUND OF THE DISCLOSURE

Hydrogen (H₂) is one of the most promising next-generation energy sources because of its abundance in nature as well as high efficiency in energy combustion with eco-friendly byproducts. However, H₂ is highly flammable and explosive (the lower flammable limit is 4%), and any leaks must be immediately detected. The U.S. Department of Energy (DOE) has set the target metrics of H₂ detection, including sensitivity (S>25% at 1% H₂), the limit of detection (LOD<0.1%), the response time (tᵣₑₛ<1 s at 4% H₂, tᵣₑₛ<60 s at 1% H₂), recovery time (t_{rec}<60 s), operating temperature (−30 to 80° C.) and gas environment (ambient air; 10-98% humidity). However, all previously-developed H₂ sensors do not meet the DOE target metrics.

### SUMMARY OF THE DISCLOSURE

To address this unmet need, a new electrochemical method to synthesize grain-boundary-rich metallic nanoparticle assemblies was developed. The nanoparticle assemblies were then used to build new high-performance hydrogen gas sensors. The grain-boundary-rich nanoparticle assemblies were synthesized by performing water electrolysis in an unbuffered solution with high metallic nanoparticle concentrations (at the μM level). The strong alkaline condition (pH>14) near the cathode and the continuous generation of micron-sized gas bubbles in the electrolytic cell were important for assembling metallic nanoparticles into a nanoparticle assembly with rich grain boundaries between the nanoparticles. Simple deposition of the synthesized nanoparticle assemblies onto a thermocouple forms a H₂ gas sensor. Because of the high catalytic activity of the grain-boundary-rich metallic nanoparticle assemblies, the reaction of H₂ and air takes place at room temperature in the presence of the nanoparticle assemblies. The thermocouple reads out the heat generated by the H₂ and air reaction for quantifying the H₂ concentration. The nanoparticle assemblies show an exceptional H₂ gas sensing performance at room temperature and meets nearly all the DOE target metrics, S 200% at 1% H₂, LOD=0.05%, tᵣₑₛ=7 s at 4% H₂, and 30 sat 1% H₂, t_{rec}<30 s, operating temperature=20° C. and gas environment (ambient air; 0-98% humidity). In addition to the high sensing performance, the disclosed sensors have the following unique advantages over previously-existing H₂ gas sensors. First, they do not require external power, making it highly suitable for applications in portable devices and long-term continuous monitoring. Second, the sensor fabrication is simple and low cost because it does not need new electronic components (it only requires a commercial thermometer).

The sensitivities of disclosed hydrogen sensors can be further enhanced by reducing background noise due to environmental temperature fluctuations by providing a non-hydrogen sensing reference thermometer and/or by increasing tensile strength by increasing concentrations of NaBH₄ during nanoparticle (NP) generation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D. Electrosynthesis of grain-boundary-rich Pt NP assembly from colloidal Pt NPs. (1A) Schematic drawing of the NP assembly process driven by NP collisions under water electrolysis conditions that results in the formation of a macroscopic grain-boundary-rich Pt NP assembly. The local high pH near the cathode assists the citrate ligand removal from the Pt NP surface, and the electrogenerated gas bubbles promote the Pt NP collisions in solution. (1B and 1C) Photograph and STEM image of (1B) Pt NPs and (1C) Pt NP assembly. (1D) High-resolution STEM image of the grain boundaries between the Pt NP building blocks in Pt NP assembly.

FIGS. 2A, 2B. (2A) pH as a function of distance from the cathode (a Pt foil with an area of 240 mm²) at −2 V vs. Ag/AgCl reference electrode during electrosynthesis of NP assembly. pH values were measured using a micro pH electrode (Thermo Scientific, Product Number: 9863BN). The solution pH value gradually decreased from 14 near the cathode surface (less than 1 mm) to 5.3 in the bulk solution. (2B) High-resolution C1s XPS spectra of Pt NPs, pH=14 treated Pt NPs (Pt NPs were precipitated by adding sodium hydroxide aqueous solution until solution pH reaches 14), and Pt NP assembly. The peak centered at 288 eV belongs to the C═O surface functional group of sodium citrate ligands (Palmisano et al., Int. J. Hydrogen Energy 39, 20491-20496 (2014)). The results indicate that most sodium citrate ligands were removed from the Pt NP under the electrogenerated highly alkaline environment near the cathode.

FIG. 3. Pt NP assembly process. Ex-situ TEM images of Pt NP solution near the cathode as a function of time. After applying a potential of −2 V vs. Ag/AgCl at the cathode for 0 min, 2 min, 5 min, 10 min, 30 min, 45 min, 60 min, 90 min, 120 min, and 180 min, 10 μL of Pt NP solution was sampled from the region that was extremely close to the cathode surface using a pipette at each time point. Then, the collected Pt NP solutions were drop-casted on the TEM grids (Ted Pella, Product Number: 01800-F) and naturally dried in the air before TEM analysis.

FIG. 4. Crystallite size distributions of Pt NPs and Pt NP assembly. The distributions were obtained by counting 100 individual particles or crystallites in the corresponding TEM images. The average diameters of Pt NPs and Pt NP assembly are 3.6 nm and 3.8 nm, with standard deviations of 0.5 nm and 0.7 nm, respectively.

FIG. 5. Identification of the grain boundaries in the Pt NP assembly based on angles between the Fourier-transform patterns of two neighboring crystals.

FIGS. 6A, 6B. (6A) Identification of a Σ11 and (6B) a highly-disordered grain boundary in the Pt NP assembly based on angles between the Fourier-transform patterns of two crystals.

FIGS. 7A, 7B. Photographs of Pt NP assemblies. (7A) Wet Pt NP assembly dispersed in DI water. (7B) Dry Pt NP assembly powder after critical point drying. There was no noticeable volume loss of the Pt NP assembly during the critical point drying.

FIG. 8. Electrochemical surface area measurement. Glassy carbon electrode (GCE, d=3 mm), graphite rod, and Ag/AgCl (sat′d KCl) are used as the working, counter, reference electrodes, respectively. 0.1 M HClO₄ aqueous solution is used as the electrolyte. Prior to use, GCE was consecutively polished by 1 μm, 0.3 μm, and 0.05 μm Al₂O₃ powders and then washed by sonication in 0.1 M H₂SO₄ and 1:1 (v:v) H₂O/ethanol mixture. The Pt black and Pt NP inks were prepared by sonicating a mixture of 1 mg Pt material, 4 mg Vulcan carbon, and 1 mL 3:1 (v:v) H₂O-isopropanol (IPA) with 0.05% Nafion for 30 min. The preparation of Pt NP assembly ink was slightly different. Briefly, 1 mg Pt NP assembly powder was added to 1 mL 3:1 (v:v) H₂O-IPA and sonicated for 15 min. Afterward, 2 mg Vulcan was added to the above suspension and sonicated for another 10 min, followed by adding 10 μL 5% Nafion and another 5 min sonication. 3 μL of ink was drop-cast by a pipette onto the GCE and dried in air, resulting in a mass loading of 3 μg Pt on the electrode or 42.5 μg/cm²_{geo}. Cyclic voltammograms of Pt NPs, Pt NP assembly, and Pt black were recorded at a potential window between −0.25 V and 1.0 V at a scanning rate of 50 mV/s. All the measurements were performed three times to obtain the average values with standard deviations.

FIGS. 9A-9G. Catalytic hydrogen sensing performance of Pt NP assembly at room temperature. (9A) Temperature readout (T) of the catalytic hydrogen sensors built using Pt NPs (blue dots) and Pt NP assembly (orange dots) in response to stepwise decreasing H₂ concentrations (CH₂) from 4% to 0.05% in air at room temperature. The sensor temperature was recorded at 1 point per second. Insert: photograph of a catalytic hydrogen sensor which is a J-type thermocouple coated with Pt NPs or Pt NP assembly, and an expanded view of the temperature response curves. (9B) Sensor response (ΔT=T−T_{ambient}, where T_{ambient} is the ambient temperature) as a function of CH₂ for Pt NPs (blue dots) and Pt NP assembly (orange dots). (9C) Response time (tᵣₑₛₚₒₙₛₑ) and recovery time (t_{recovery}) of the catalytic hydrogen sensor based on Pt NP assembly at various CH₂. (9D to 9E) Measured ΔT as a function of CH₂ when the Pt NP assembly-based sensor was operated at (9D) an ambient temperature range from −30° C. to 80° C. and (9E) a humidity range from 0% to 98%. (9F) The percentage change of sensor response when exposed to 36 different interference gases (including CO, NO₂, SO₂, H₂S, NH₃, and various organic gases and vapors such as formaldehyde, methanol, etc.) in the absence and presence of 4% H₂ in air. The concentrations of the interference gases are 4%, except for NO₂ and SO₂ (4 ppm), ethylene (40 ppm), and H₂S (0.4 ppm). Error bars denote the standard deviations of at least three independent measurements. (9G) Stability of the Pt NP assembly-based hydrogen sensor during a 280-hour-long 288-test-cycles test. In each cycle, CH₂ was decreased from 4% to 0.05% stepwise as in (9A).

FIG. 10. Catalytic H₂ sensing performance of various Pt materials. Response-recovery curves of a J-type thermocouple coated with 1 mg of various Pt catalysts in response to H₂ at different concentrations (4%-0.05%) at room temperature. Pt NPs (precipitated by centrifuge), Pt NPs without ligands (prepared by adjusting the solution pH to 14 using a NaOH solution, see FIGS. 2A, 2B), Pt NP assembly, Pt black (Sigma Aldrich), Pt bulk powder (Sigma Aldrich), Adams' catalyst (Sigma Aldrich) were separately mixed with ethanol to prepare a catalyst slurry which was drop-casted on the tip of J-type thermocouple, followed by drying under the ambient condition to prepare the catalytic H₂ sensors.

FIGS. 11A-11C. One-second H₂ sensing test. (11A) Temperature variations of a Pt NP assembly sensor in response to 1-second-long pulses of H₂ with various concentrations from 4% to 0.05% for 23 cycles. (11B) Expanded view of the first cycle result where sensor temperature data was recorded at one point per second. (11C) Statistics of sensor temperatures upon exposure to different H₂ concentrations for 1 s.

FIGS. 12A-12D. Operating temperature (12A) and humidity (12D) dependence tests. High ambient temperatures (50° C. and 80° C.) were created by heating the sensing test Teflon chamber (12B) where the J-type thermocouple was housed, while the ultra-low temperature of −30° C. was achieved by placing dry ice underneath the testing chamber (12C). The catalytic H₂ sensor was exposed to a step-wise decreasing H₂ concentration from 4% to 0.05%. For the humidity test, the relative humidity was adjusted by regulating the flow rate ratio of dry air and wet air (bubbling through DI water). The sensor was exposed to H₂ concentrations from 4% to 0.05% at the humidity level of 0%, 25%, 50%, 75% and 98% at room temperature.

FIG. 13. Interference/cross-sensitivity tests. Thirty-six interference gases were tested. Their concentrations were set at 4% with only a few exceptions, including 0.4 ppm for H₂S, 4 ppm for NO₂, 4 ppm for SO₂, and 40 ppm for ethylene. The first panel illustrates the test protocol using CO as an example. Specifically, a J-type thermocouple coated with 1 mg Pt NP assembly was first exposed to three pulses of 4% CO in air to test whether the sensor responds to CO at room temperature, then three pulses of 4% CO but in the presence of 4% H₂ to access the cross-sensitivity, finally three pulses of 4% H₂ to test if there is irreversible poisoning.

FIG. 14. Long-term stability test. In each cycle, the Pt NP assembly sensor was exposed to H₂ concentrations from 4% to 0.05% at room temperature. 10 cycles were conducted per day for one month to complete 288 cycles with a total testing time of 281 h.

FIGS. 15A-15G. Universal applicability. Photographs of catalytic H₂ sensors prepared from (15A) liquid-in-glass thermometer; (15B) J-type thermocouple/multimeter (Tekpower, Part number: TP4000ZC); (15C) K-type thermocouple/multimeter; (15D) pocket digital thermometer; (15E) cooking thermometer; (15F) thermistor (inside the A9 rectangle, dimension: 1 mm×2 mm). The thermistor in a circuit playground (Adafruit, Product Number: 2769) was connected to a PC by a CircuitPython Program for recording the temperature. For all experiments, the thermometer was loaded with 1 mg Pt NP assembly and exposed to 1% H₂ in air at room temperature. (15G) The responses of the above six catalytic H₂ sensors at H₂ concentrations from 0.05% to 4% at room temperature. 1 mg Pt NP assembly costs less than $1, and the liquid-in-glass thermometer is $3 (Newark.com, #13AJ1664, $2.38 each), making the total cost of a sensor<$5 per unit.

FIGS. 16A-16C. H₂ leak detection demonstration using a drone sensing platform. 1 mg Pt NP assembly was deposited on the tip of a K-type thermocouple connected to a circuit playground (Adafruit, Product Number: 2769) with a thermocouple amplifier (Adafruit, Product Number: MAX31855) on a commercial drone (DJI Mini). The circuit playground was wirelessly connected to a smartphone by Bluetooth to report the temperature in real-time. H₂ gas was released to the hood via a tube to simulate a H₂ leakage site. Photographs of (16A) the drone H₂ sensing platform, (16B) the drone above the H₂ leakage site, and (16C) sensor temperature readout during the test.

FIGS. 17A-17G. Structural characterization of the grain boundaries in Pt NP assembly. (17A, 17B) HRTEM images of (17A) Pt NP and (17B) Pt NP assembly near a Σ3 grain boundary. (17C, 17D) The integrated pixel intensities of Pt NP and Pt NP assembly along (17C) (111) and (17D) (200) spacing directions. The peaks and valleys represent the atoms and gaps, respectively. The spacings of Pt (111) and Pt (200) planes were averaged over 3 atomic layers for high accuracy as labeled in (17A) and (17B). The lattice expansion at the Σ3 grain boundary along the (111) direction leads to an offset between the intensity profiles of Pt NP and Pt NP assembly in (17C). (17E) Statistics of d spacings for the (111) and (200) facets, analyzed under the TEM pixel size of 0.0149 nm, shows Pt NP assembly has similar mean value as Pt NP assembly but a broader distribution of d spacings than NPs (for (111), standard deviation: 0.021 vs 0.017 nm; for (200), standard deviation: 0.019 vs 0.015 nm). (17F) High-resolution XRD spectra of Pt NPs and Pt NP assembly showing the (111) and (200) diffraction peaks. (17G) Full width at half maximum (FWHM) of the XRD peaks for Pt NPs and Pt NP assembly.

FIGS. 18A-180. X-ray absorption spectroscopy results. (18A) k-space k³-weighted Pt L3-edge extended X-ray absorption fine structure (EXAFS) and (18B) Fourier-transformed EXAFS data (circles) and theoretical fits (lines) of Pt foil, Pt black, Pt NPs, Pt NP assembly. (18C) Coordination numbers (CN), Debye-Waller factors (ρ²), and inter-atomic distances (R) were obtained from the data fitting.

FIGS. 19A-19D. High-resolution x-ray diffraction (XRD) spectroscopy results. The high-resolution XRD spectra of (19A) Pt NPs, (19B) Pt NP assembly, and (19C) Pt black were fitted using Psdvoigt 1 function in OriginPro Software into eight peaks corresponding to eight planes of (111), (200), (220), (311), (222), (400), (331), and (420). The fitted peak widths at half maximum for the three samples were summarized in (19D).

FIGS. 20A-20D. Effect of grain boundary on the catalytic hydrogen oxidation performance. (20A) High-resolution STEM image of a Σ3 grain boundary between two Pt NPs in Pt NP assembly and its strain mapping near the grain boundary. (20B, 20C) Calculated strain distributions in (20B) an isolated Pt NP containing 1925 Pt atoms and (20C) two Pt NPs with the same size but connected by a Σ3 grain boundary. The strain values are referenced to Pt (111) bulk. (20D) Calculated reaction rate vs strain in Pt using strained Pt (111) surface models.

FIG. 21. Grain-Boundary energy may vary between individual grain-boundaries within a Pt NP assembly.

FIGS. 22A-22D. Baseline subtraction for improving sensing performance. (22A) Photograph and an expanded view (22B) of two J-type thermocouples coated with (left) and without (right) Pt NP assembly catalyst. (22C) Temperature readout (T) of two thermocouples with (green dots) or without (grey dots) Pt catalyst in response to stepwise decreasing H₂ concentrations (CH₂) from 500 ppm to 12.5 ppm in air at room temperature. The sensor temperature was recorded at 1 point per second. (22D) Temperature readout after substrating the baseline signal recorded by the reference thermocouple without catalyst from the sensor signal.

FIGS. 23A-23E. Structural characterization of the tensile-strained Pt catalysts. The Pt catalysts denoted as 1:1(20 mL), 1:1(15 mL), 1:1(10 mL), and 10:1 were synthesized by adding 10 mL of 0.01M NaBH₄ into 10 mL of 0.01 M Na₂PtCl₆, 7.5 mL of 0.013M NaBH₄ into 7.5 mL of 0.013 M Na₂PtCl₆, 5 mL of 0.02M NaBH₄ into 5 mL of 0.02 M Na₂PtCl₆, and 5 mL of 0.2M NaBH₄ into 5 mL of 0.02 M Na₂PtCl₆, respectively. After synthesis, the samples were washed with DI water three times by centrifuge. The Pt foil and Pt black were purchased from a commercial supplier. (23A) X-ray diffraction patterns showing the (111), (200), (220), (311), and (222) diffraction peaks of the Pt catalysts. (23B) Expanded view of the (111) XRD diffraction peak. (23C) The d-spacing of five planes is calculated using the Bragg diffraction equation. TEM images of the 1:1(20 mL) Pt sample (23D) and 1:1 (10 mL) one (23E).

FIGS. 24A-24B. (24A) Temperature readout (T) of the Pt catalysts in response to stepwise decreasing H₂ concentrations (CH₂) from 500 ppm to 25 ppm in air at room temperature. The temperature readout was measured using a thermocouple coated with Pt catalyst subtracting the background signal monitored by a thermocouple without catalyst. The sensor temperature was recorded at 1 point per second. (24B) The relationship between tensile strain and temperature variation.

### DETAILED DESCRIPTION

Hydrogen gas (H₂) sensors play an important role in the hydrogen economy due to the safety concerns associated with the production, distribution, and utilization of flammable H₂. The United States Department of Energy (DOE) has set targets for H₂ sensors, in terms of concentration range (0.1-10%), operating temperature (−30 to 80° C.), response time (<1.0 s), gas environment (ambient air; 10-98% humidity), and lifetime (>10 years). (U.S. Department of Energy, Energy Efficiency and Renewable Energy (EERE), Fuel Cell Technologies Office. Multi-Year Research, Development, and Demonstration Plan, 2011-2020. Section 3.7 Hydrogen Safety, Codes and Standards (EERE, 2015)) In addition to these metrics, H₂ sensors should be resistant to poisoning gases (e.g., hydrocarbons, CO, NO₂, H₂S, SOₓ), (Palmisano et al., Int. J. Hydrogen Energy 40, 11740-11747 (2015); Palmisano et al., Int. J. Hydrogen Energy 39, 20491-20496 (2014)) inexpensive (<$40 per unit), (Koo et al., Chemiresistive ACS Nano 14, 14284-14322 (2020)) miniaturized, and power-efficient (<1 W), (Darmadi, Nugroho, LanghammerACS Sensors 5, 3306-3327 (2020)) to ensure their effectiveness in various applications. There have been enormous efforts to develop high-performance and efficient H₂ sensors; however, it is incredibly challenging to meet all the target metrics. (Koo et al., Chemiresistive ACS Nano 14, 14284-14322 (2020); Darmadi, Nugroho, LanghammerACS Sensors 5, 3306-3327 (2020); Hubert, et al., Chem. 157, 329-352 (2011); Penner, Acc. Chem. Res. 50, 1902-1910 (2017); Nugroho et al., Nat. Mater. 18, 489-495 (2019); Favier, et al., Science 293, 2227-2231 (2001); Yang, et al., ACS Nano 4, 5233-5244 (2010)).

Typical commercially available H₂ sensors are of four types: electrochemical, catalytic, metal-oxide-semiconductor, and thermal conductivity, claiming over 90% of the H₂ sensor market. (Boon-Brett, Bousek, Moretto, Int. J. Hydrogen Energy 34, 562-571 (2009); Boon-Brett et al., Int. J. Hydrogen Energy 35, 373-384 (2010)) Among the four sensor types, catalytic sensors were found to be the most accurate and robust sensor type because they show little to no dependence of sensor output on the ambient parameters such as temperature, pressure, and humidity. (Boon-Brett, Bousek, Moretto, Int. J. Hydrogen Energy 34, 562-571 (2009)) Catalytic sensors detect H₂ based on the exothermic oxidation reaction of H₂ with air on a catalyst surface (typically, Pt). The exothermic oxidation of H₂ with air generates heat and changes the electrical resistance of the catalyst, enabling the H₂ concentration to be determined. However, due to the low activity of Pt catalysts in conventional catalytic H₂ sensors, a high operating temperature of 500° C. is often required, causing high power consumption (0.5-3.0 W) and cross-sensitivity to other combustible gases, such as hydrocarbons, CO, etc, (Palmisano et al., Int. J. Hydrogen Energy 40, 11740-11747 (2015), Boon-Brett, Bousek, Moretto, Int. J. Hydrogen Energy 34, 562-571 (2009); Boon-Brett et al., Int. J. Hydrogen Energy 35, 373-384 (2010); Hubert, et al., Chem. 157, 329-352 (2011)) In addition, the performance of catalytic H₂ sensors is also affected following exposure to poisoning gases, such as H₂S and SO₂ (even at ppm level), which adsorb onto the Pt catalyst more strongly than H₂, thereby reducing the number of sites available to catalyze H₂ oxidation. (Hubert, et al., Chem. 157, 329-352 (2011)) To overcome the limitations of catalytic H₂ sensors, prior efforts focused on the miniaturization of the sensors, resulting in significantly lower power consumption and faster response times. (Lee et al., Sensors and Actuators, 153, 392-397 (2011); Harley-Trochimczyk et al., Adv. Funct. Mater. 26, 433-439 (2016); Del Orbe Henriquez et al., ACS Applied Nano Materials 4, 7-12 (2021); Liu, Dong, Xia, Micro Nano Lett 8, 668-671 (2013); Cavicchi et al., Chem. 97, 22-30 (2004); Harley-Trochimczyk et al., Chem. 206, 399-406 (2015)) However, the cross-sensitivity to combustible gases and susceptibility to catalyst poisoning is still not addressed.

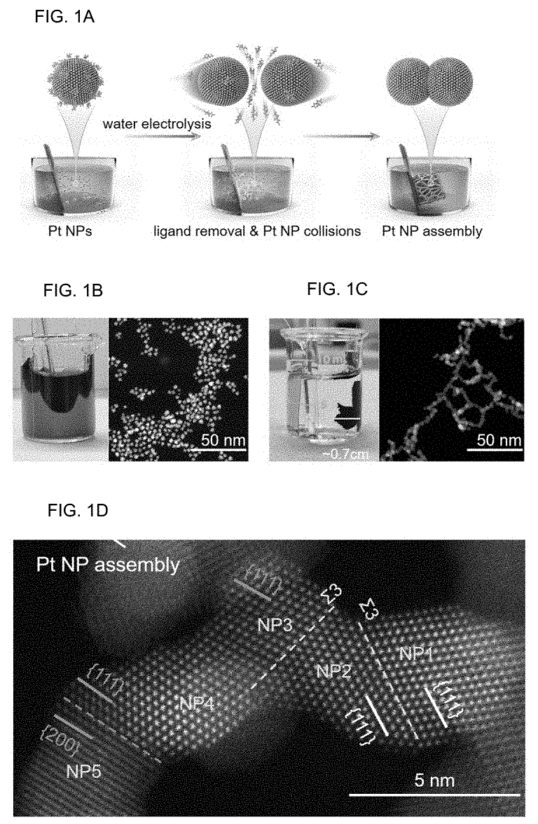

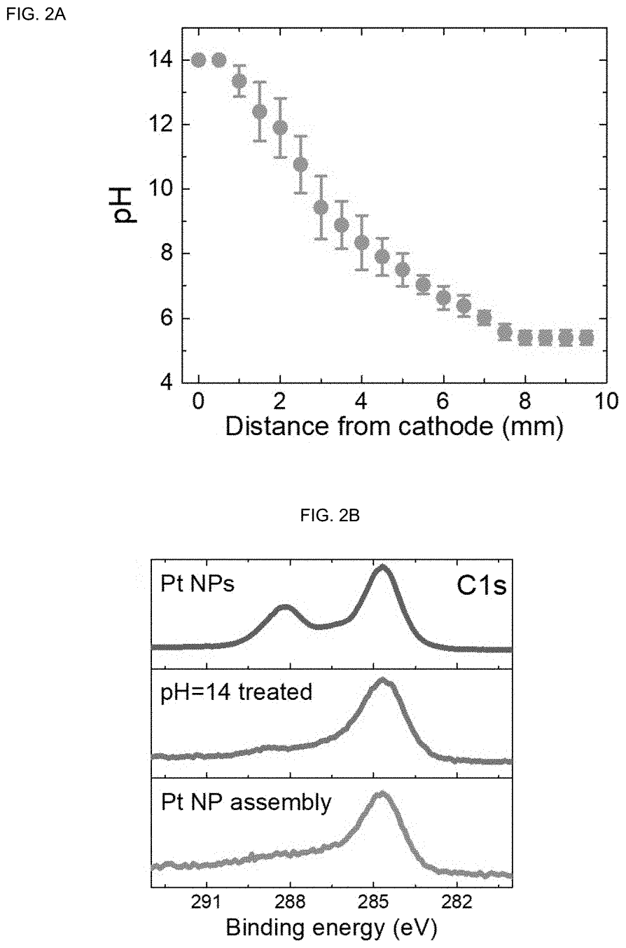

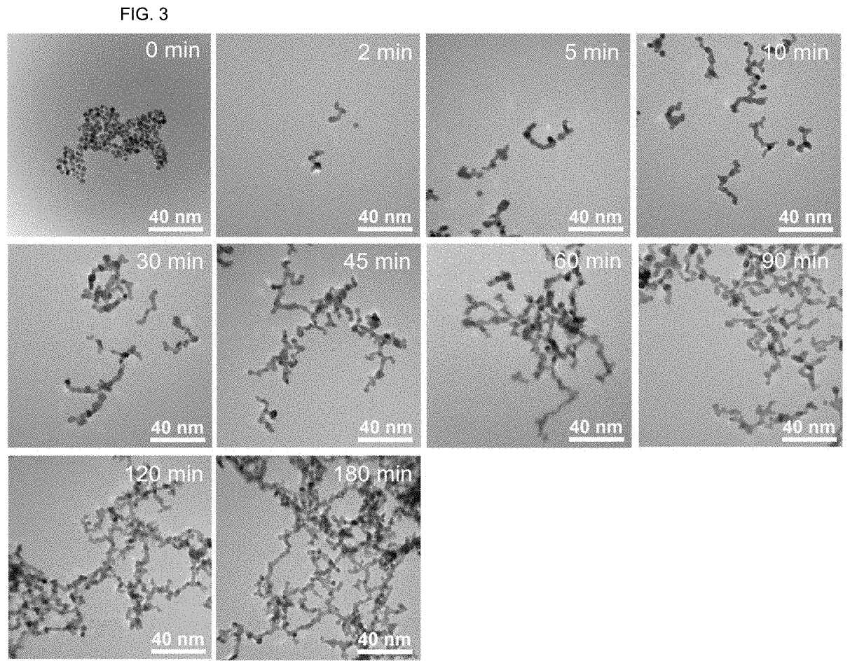

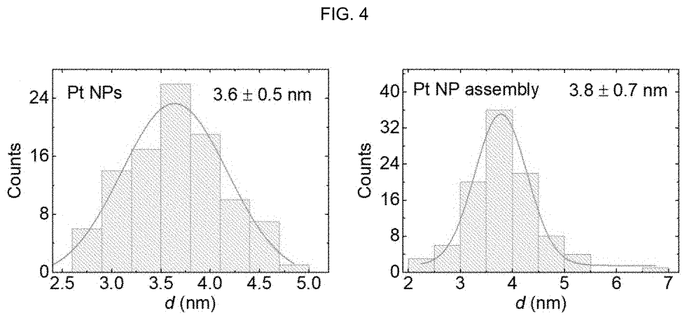

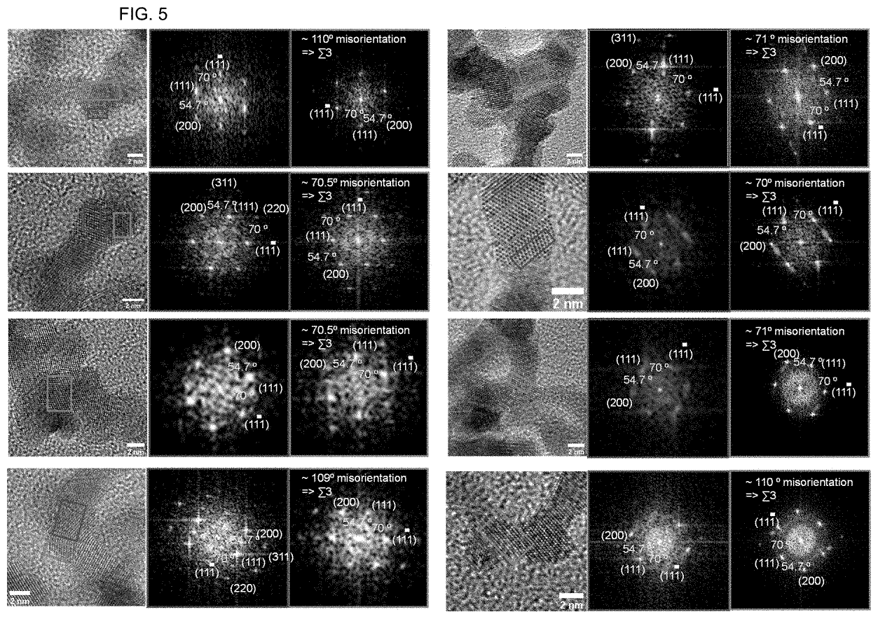

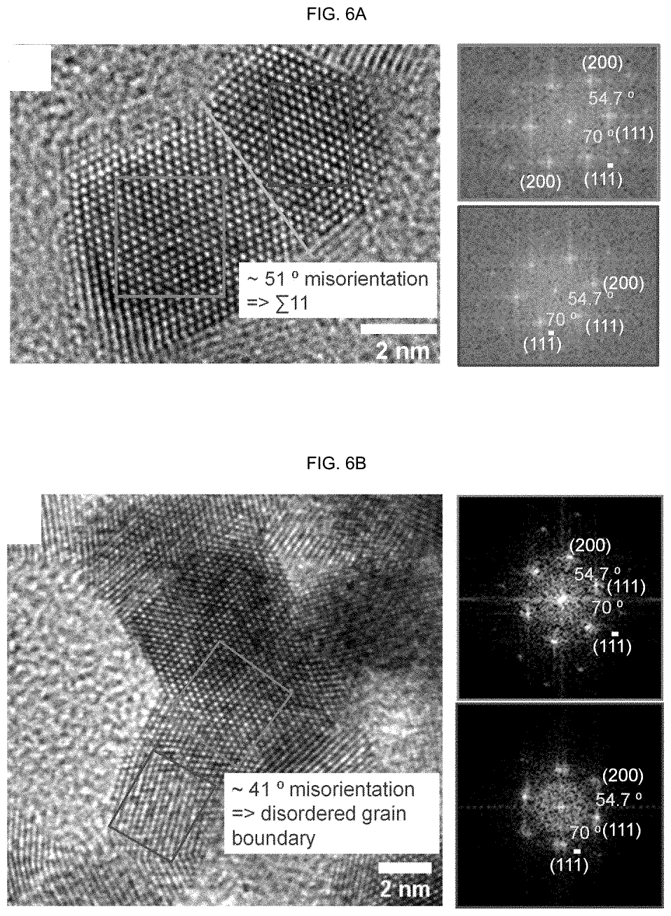

Here, a grain-boundary-rich Pt nanoparticle (NP) assembly material that catalyzes H₂ oxidation in air at room temperature and exhibits low cross-sensitivity and negligible poisoning effect to common pollutant gases in the atmosphere (e.g., CO, NO₂, SO₂, H₂S, and NH₃) and various combustible organic gases and vapors (e.g., natural gas, alkanes, alkenes, alcohols, ketones, aldehydes, ethers, aromatic compounds, amine, and acetates) is described. While the nanoparticle assembly can be formed from Pt nanoparticles, in some additional examples, the nanoparticle assembly can be formed from metallic nanoparticles that provide catalytic activity for H₂ oxidation (e.g., platinum (Pt), palladium (Pd), gold (Au), Silver (Ag), Ruthenium (Ru), Rhodium (Rh), alloys of the various metals, etc.). It should be noted that a grain-boundary commonly refers to a transition region between a first NP with first crystalline structure and a second NP with a second crystalline structure that have been combined. It should be noted that the first NP and the second NP can be monocrystalline nanoparticles (e.g., the first Pt NP contains only the first crystalline structure) and/or polycrystalline nanoparticles (e.g., the first NP contains the first crystalline structure and one or more additional crystalline structures). Additionally, and as described in greater detail elsewhere herein, the grain-boundary can be formed during assembly of the first NP and the second NP. In certain examples, the transition region of the grain-boundary is formed when assembly of the first NP and the second NP cause the first crystalline structure and the second crystalline structure to lose definition and combine such that the first NP and the second NP adhere to each other. As illustrated in FIG. 1A, a grain-boundary-rich NP assembly can be synthesized by performing water electrolysis in an unbuffered, high-concentration (1.7 μM) colloidal metallic (e.g., Pt) NP solution. In certain examples, a high-concentration colloidal NP solution is at least 1.0 μM, at least 1.5 μM, at least 2.0 μM, or at least 2.5 μM. A cathode within the colloidal NP solution can assist in the removal of capping agents (e.g., citrate capping agents and/or other capping agents) from the NP surface by providing a basic pH (e.g., a local high pH of 12, 13, or 14 can exist at the cathode, with in certain examples the local high pH being 14) (FIG. 2). Gas bubbles generated during water electrolysis promote random collisions of the destabilized NPs after ligand removal (e.g., removal of the capping agents) and drive individual NPs to assemble into NP clusters, chains, networks, and eventually can form a macroscopic (sub-centimeter-sized) solid (FIGS. 1B, 1C, 3) nanoparticle assembly. Scanning transmission electron microscopic (STEM) images of Pt NPs and Pt NP assembly show no significant crystallite size change after the water-electrolysis-driven NP assembly (Pt NPs: 3.6±0.5 nm and Pt NP assembly: 3.8±0.7 nm, FIG. 1B-1C, 4). High-resolution TEM (HRTEM) and STEM images revealed that individual Pt NP building blocks in the Pt NP assembly were primarily connected by grain boundaries (FIG. 1D). The representative grain boundaries identified in the Pt NP assembly are Σ3 twin grain boundaries (FIG. 5), which are formed along the low-energy {111} planes of two NPs (e.g., NP1 and NP2 in FIG. 1D). (Song et al., Science 367, 40 (2020); Grimmer, Bollmann, Warrington, Acta Crystallographica Section A 30, 197-207 (1974)) Σ11 grain boundaries (FIG. 6A) as well as highly-disordered grain boundaries (e.g., the grain boundaries between the {111} plane of NP4 and the {200} plane of NP5 in FIG. 1D and FIG. 6B) were also observed. It is worth noting that an interface-free structure can also form when two NPs attach along the {111} planes (e.g., NP3 and NP4 in FIG. 1D).

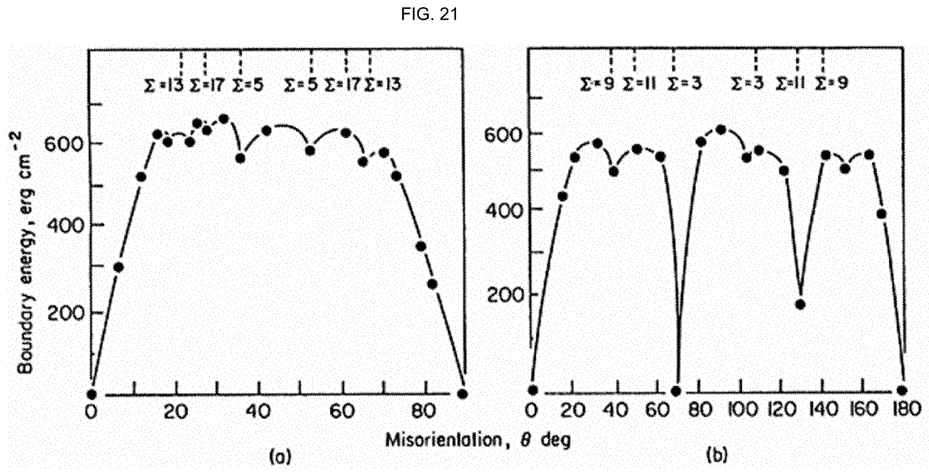

Without being bound by theory, the low-energy planes of the Pt NPs may be associated with the formation of the grain-boundaries. In particular, the Pt NPs may assemble to form the Pt NP clusters, chains, networks, and ultimately, the Pt NP assembly when collisions between Pt NPs enable the grain-boundaries to form between low-energy planes of the Pt NPs. It should be noted that wain-boundaries may form at high-energy planes of the Pt NPs, but formation of grain-boundaries at high-energy planes may occur at a formation rate different than that of the low-energy planes. It should be noted that planes of the NPs can refer to crystalline structure(s) of the NPs that form during synthesis of the Pt NP. Additionally, the highly-disordered grain boundary can form as a high-angle grain boundary or a low-angle grain boundary based at least on an angle of contact between a first NP and a second NP during synthesis of the NP assemblies. More specifically, the highly-disordered grain boundary can form when the first NP and the second NP collide under electrolysis and coalesce to form a cluster, chain, and/or network, to form an NP assembly. During the collision, a first crystalline structure of the first NP and a second crystalline structure of the second NP may be unaligned. Accordingly, the first crystalline structure and the second crystalline structure contact at the angle and the highly-disordered grain boundary may form at the point of contact between the first NP and the second NP. Further, the highly-disordered grain boundary may be categorized based at least on a sigma value. For example, a Σ3 grain boundary (sigma value equals 3) may form where a {111} plane of a first NP collides with and connects to a {200} plane of a second NP. However, additional grain boundaries may form between different planes, the additional grain boundaries being associated with the sigma value corresponding to the planes that join at the individual grain boundaries (FIG. 21).

Without being bound by theory, the grain-boundaries may be associated with the active sites of the NP assembly that catalyze the H₂ oxidation reaction. For example, the grain-boundary between a first NP and a second NP may cause the active sites to form within the highly-disordered state of the grain boundaries. Alternatively, or in addition, the grain boundaries may activate the active sites of the NPs and enable elevated catalytic activity for the NP assembly that is greater than the NPs utilized to form the NP assembly. However, formation of the grain boundaries results in the NP assemblies exhibiting increased catalytic activity. Further, the formation of grain boundaries and the sigma value of the grain boundaries may be modified by selectivity for NP morphology (e.g., the three-dimensional arrangement of atoms/molecules within the NP and the crystalline structure of the nanoparticle) and NP structure (e.g., NPs may be formed from an alloy of two substances with consistent distribution, while other NPs may have a core of a first substance and a shell of a second substance). More specifically, NP morphology and NP structure may modify the planes available to form the grain boundaries and alter the type of grain boundaries that form between NPs (e.g., different sigma values are associated with different types of grain boundaries). Accordingly, modifications to the grain boundaries within the NP assembly may modify the catalytic activity exhibited by the NP assembly.

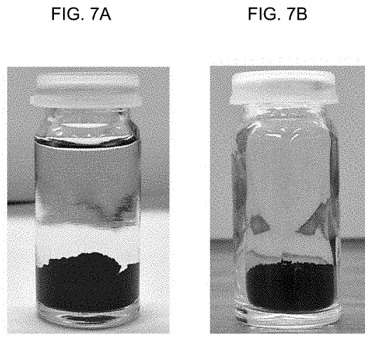

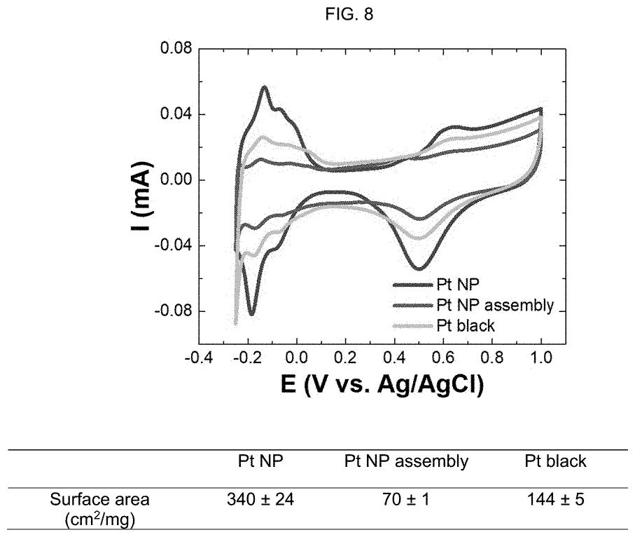

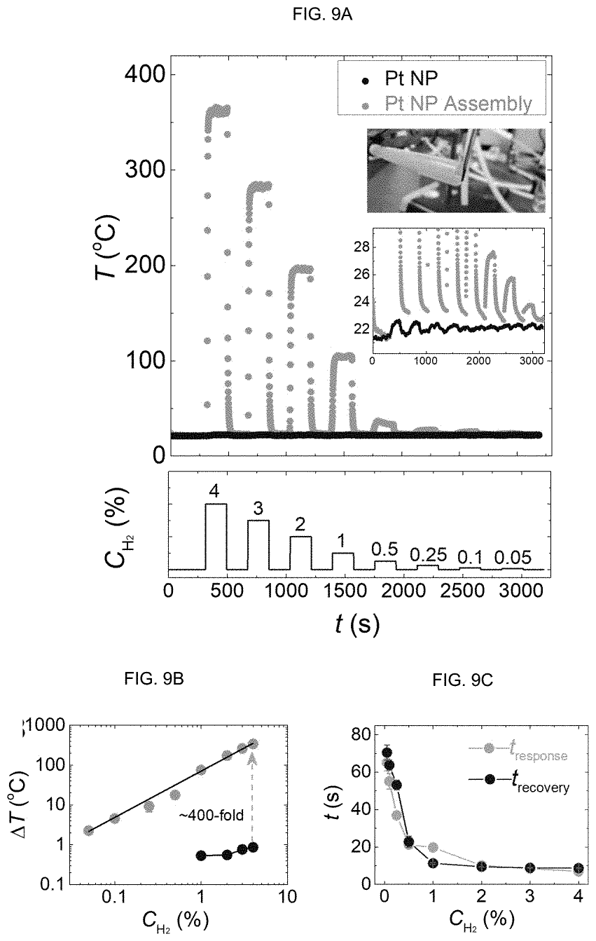

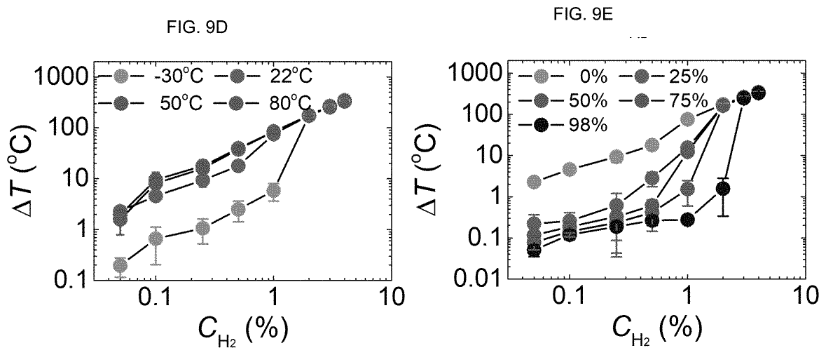

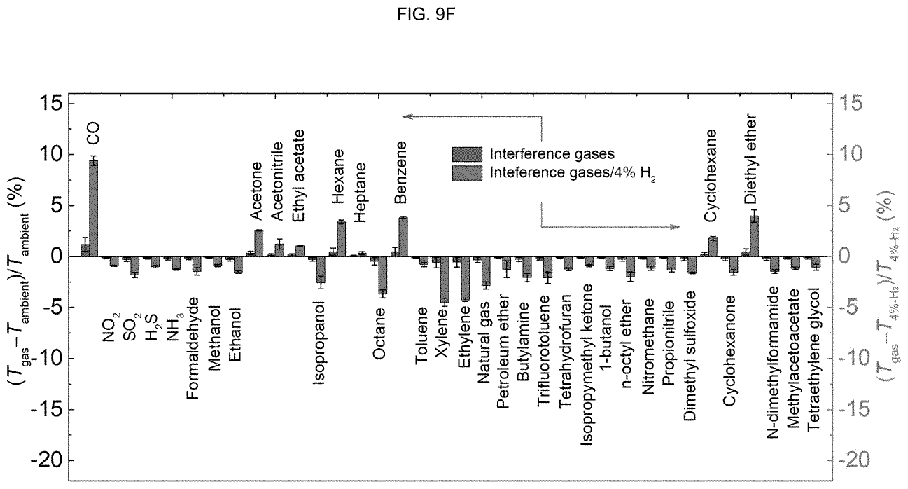

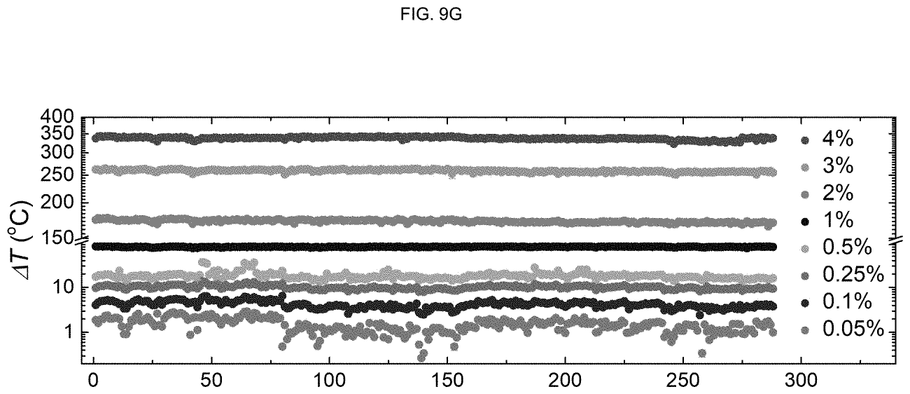

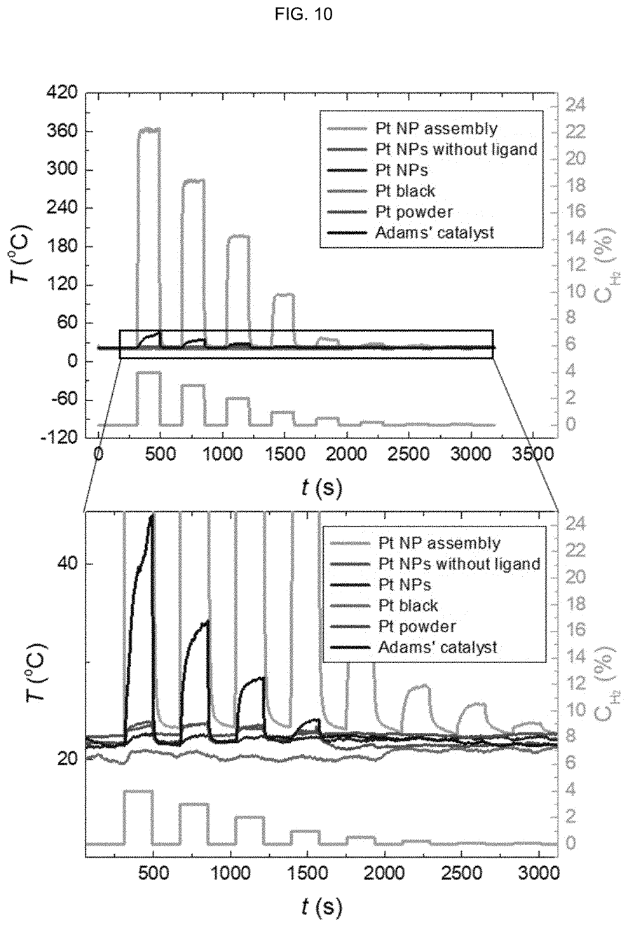

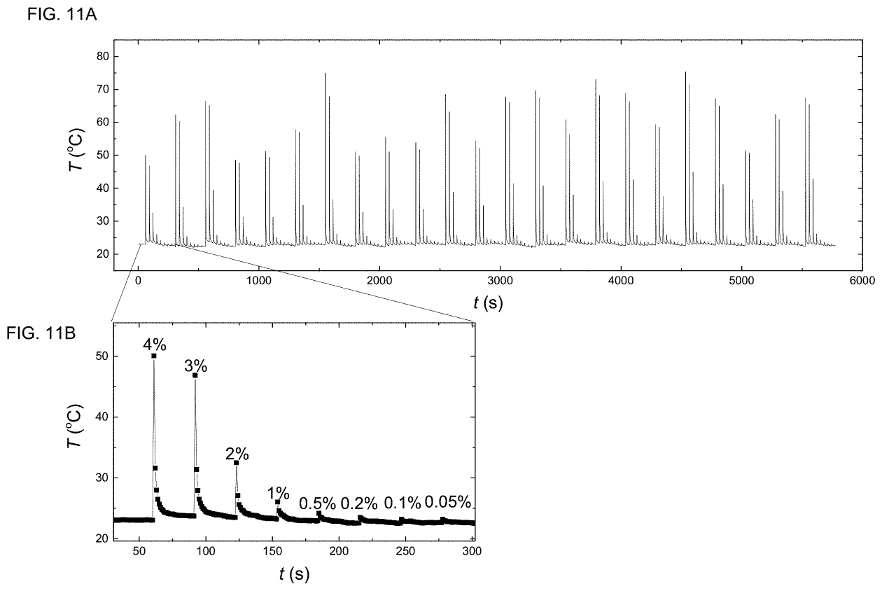

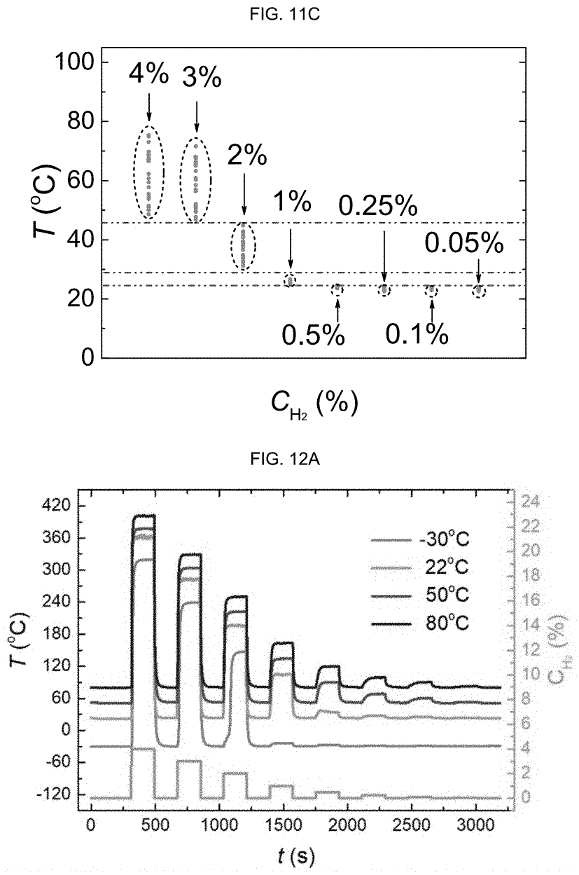

After synthesis, NP assembly can be subjected to supercritical drying to prepare a dry powder (FIG. 7). The electrochemically active surface area measured by hydrogen underpotential deposition of Pt NP assembly powder was 70±1 cm²/mg, 80% lower than that of Pt NPs (340±25 cm²/mg, FIG. 8), due to the loss of surface area when NPs are assembled. H₂ sensors can be prepared by drop-casting a mixture of Pt NP assembly powder and ethanol onto a thermocouple, followed by drying in air (FIG. 9A, insert). The sensor response is the temperature change (ΔT) of the NP assembly-coated thermocouple upon exposure to H₂. The Pt NP assembly is highly active for catalyzing H₂ oxidation in air. FIG. 9A shows that the sensor temperature rapidly increased from room temperature of 22° C. to 360° C. in 10 s, after being exposed to a stream of 4% H₂ in air (i.e., the lower explosive limit of H₂). Upon the removal of H₂, the sensor temperature quickly returned to room temperature within 10 s. ΔT decreases in response to stepwise decreasing H₂ concentrations (CH₂) from 4% to 0.05%, yielding a linear relationship between ΔT and CH₂ at the logarithmic scale (FIG. 9B). In steep contrast, when the same loading (1 mg) of Pt NPs was used to prepare a catalytic H₂ sensor, the sensor response is nearly negligible (<1° C.) even at CH₂=4% (FIG. 1A, insert, and FIG. 9B). The low catalytic activity of Pt NPs is not caused by their surface ligands blocking the active Pt sites. Control experiments were run using various Pt catalysts (FIG. 10), including ligand-free Pt black with a surface area of 144±5 cm²/mg (FIG. 8), Pt powder, Pt NPs after ligand removal, and Adams' catalyst (PtO₂). None of them shows similar or even close catalytic activity to Pt NP assembly (FIG. 10), suggesting the high catalytic activity of Pt NP assembly originates from a high concentration of grain-boundaries within the Pt NP assembly structure.

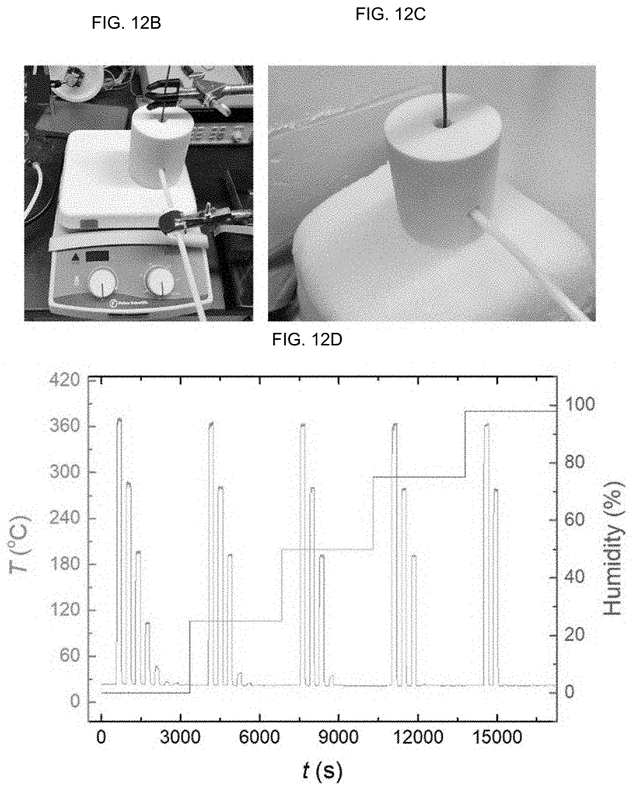

The response time and the recovery time of a Pt NP assembly sensor, defined as the time to reach 90% of the total signal, were 7 s at CH₂>1% and extended to 1 min at 0.05% (FIG. 9C). Such negative dependence of sensor response and recovery times on CH₂ is common for H₂ sensors because low CH₂ is associated with slow reaction kinetics between H₂ and the sensing material. (Koo et al., Chemiresistive ACS Nano 14, 14284-14322 (2020); Darmadi, Nugroho, LanghammerACS Sensors 5, 3306-3327 (2020); Penner, Acc. Chem. Res. 50, 1902-1910 (2017)) Even though the response time of the Pt NP assembly sensor does not meet the DOE target of 1 s, it is still comparable to, or better than, the performance of existing catalytic H₂ sensors on the market (10-30 s). (Krawczyk et al., J Autom Methods Manag Chem 25, 115-122 (2003); RAE Systems, Inc., RAE Systems Technical Note TN-114, Sensor Specifications and Cross-Sensitivities. Page 5-6) It is also important to point out that the Pt NP assembly sensor is capable of discriminating different H₂ concentrations in 1 s based on its transient temperature change (FIG. 11). Due to the self-heating effect of Pt NP assembly during catalytic H₂ sensing, the sensor is operational over a wide ambient temperature range from −30° C. to 80° C. and a humidity range from 0 to 98% (FIGS. 9D and 9E, 12). In particular, when CH₂>2%, the sensor response, response and recovery times were not affected by the ambient temperature and humidity.

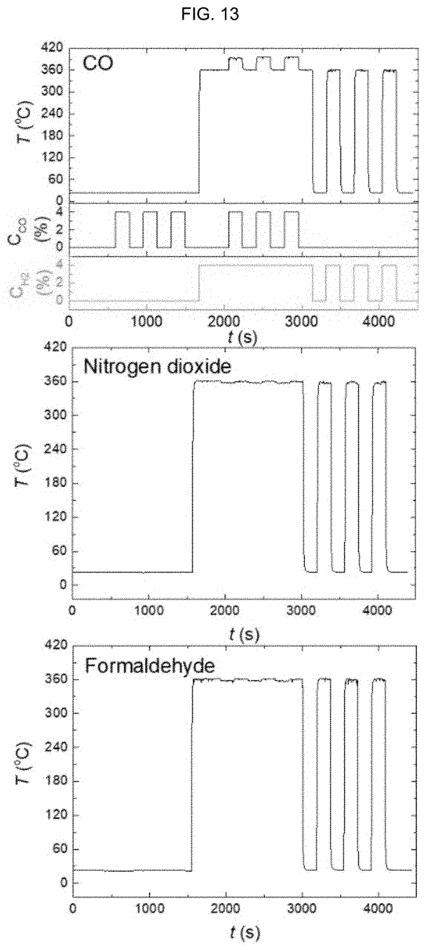

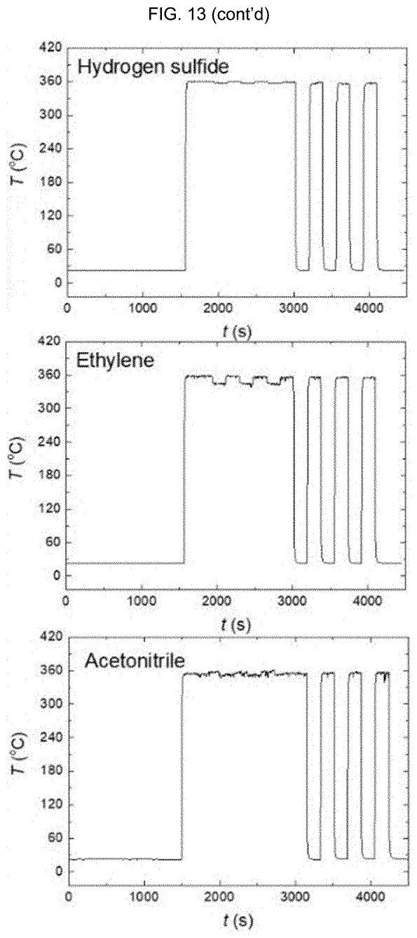

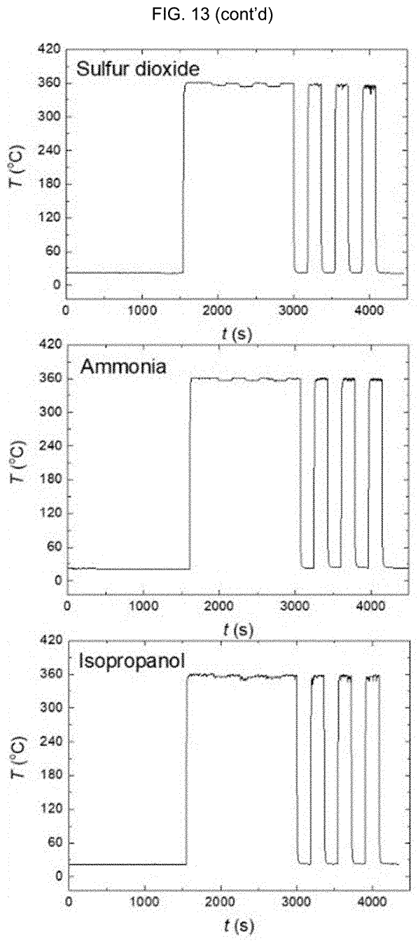

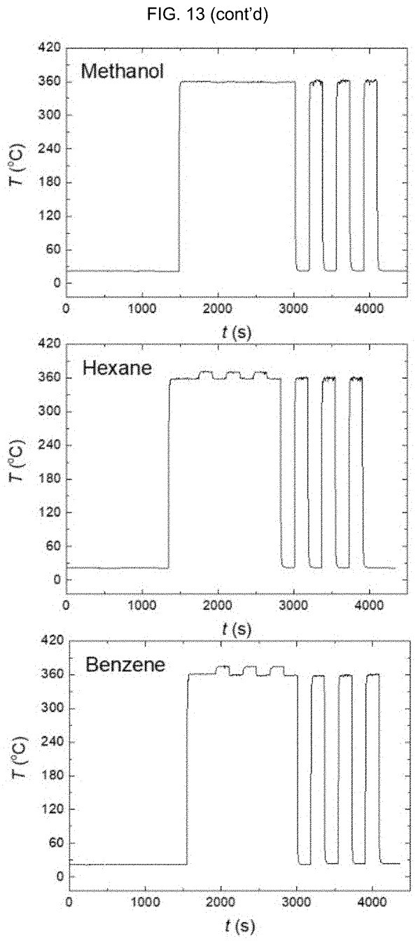

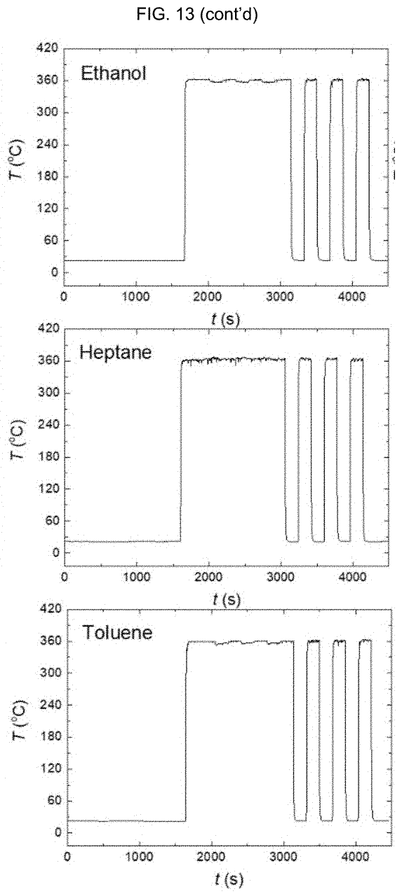

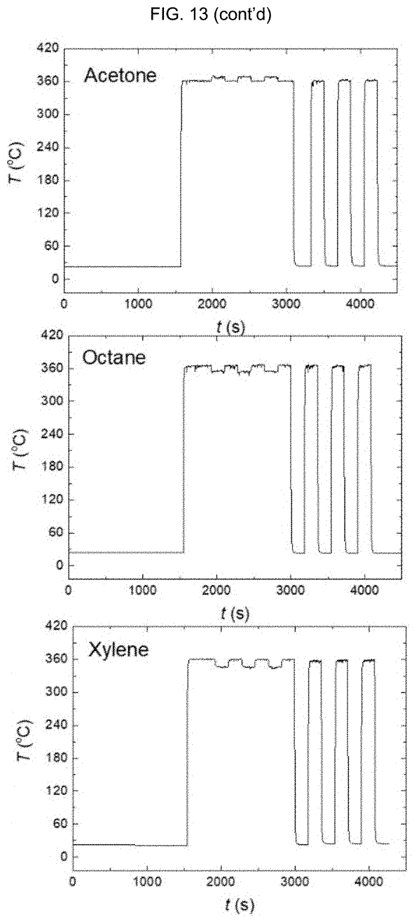

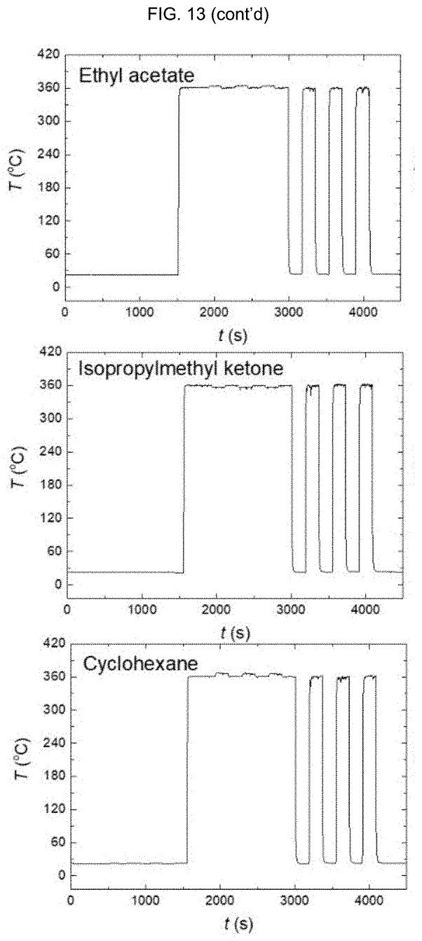

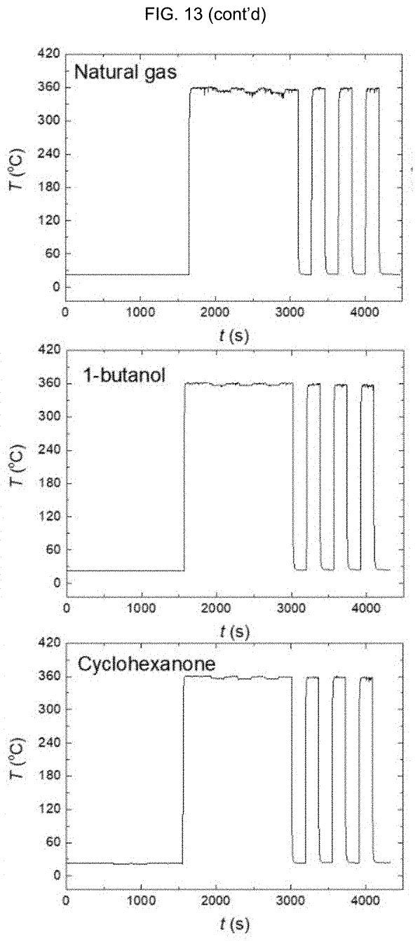

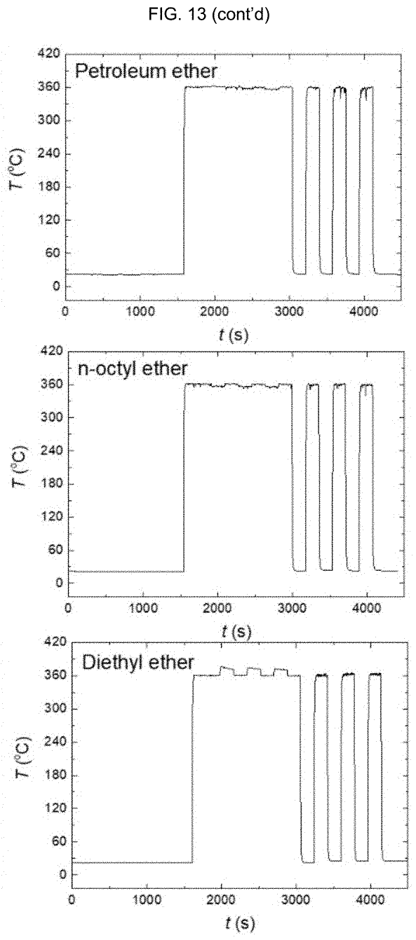

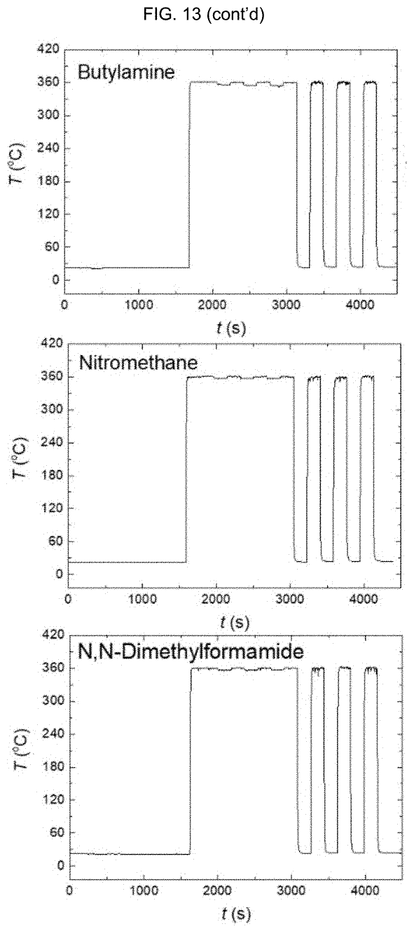

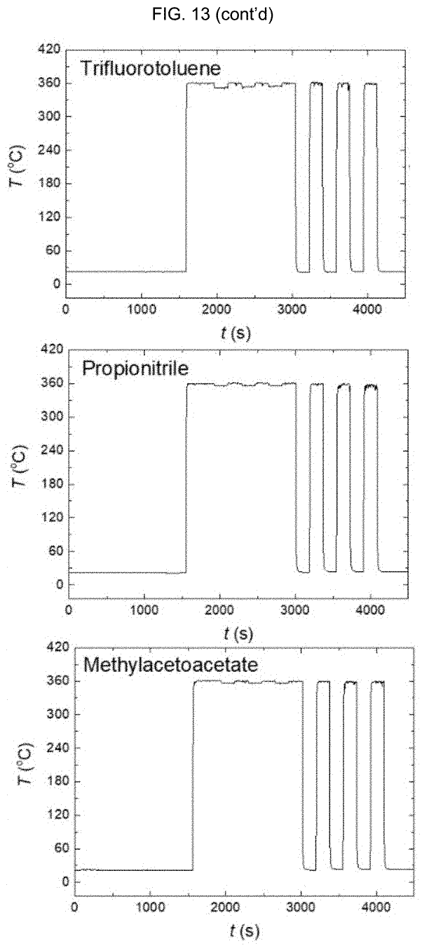

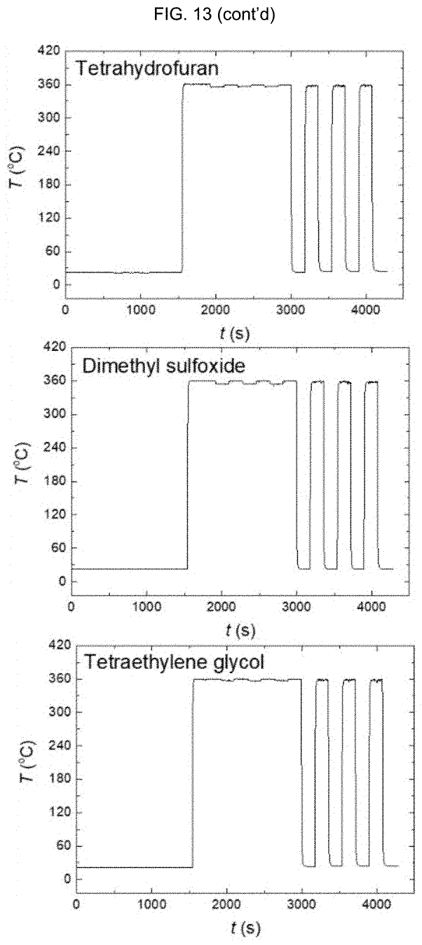

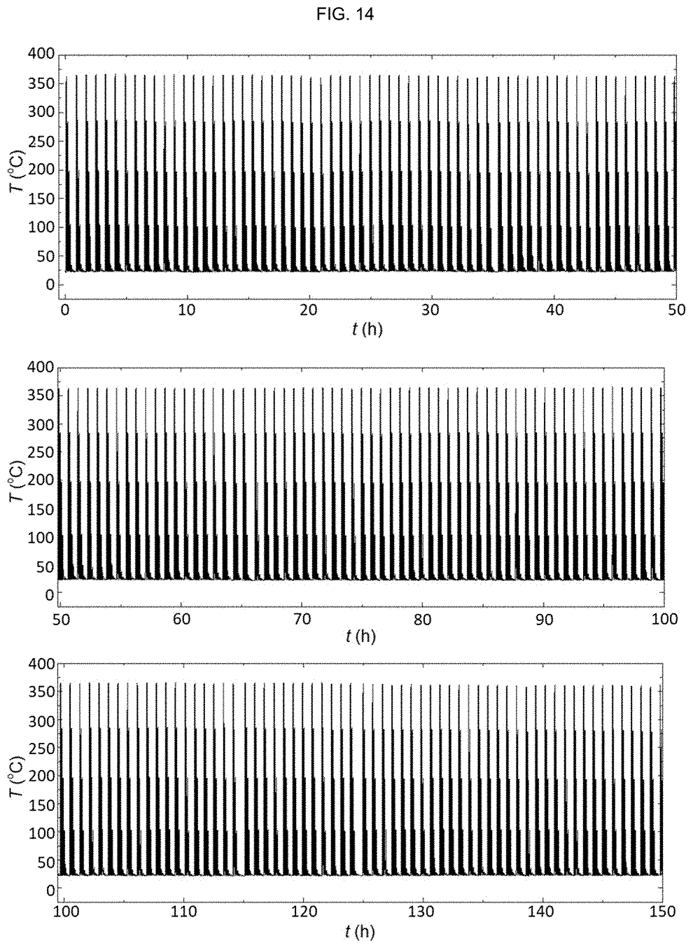

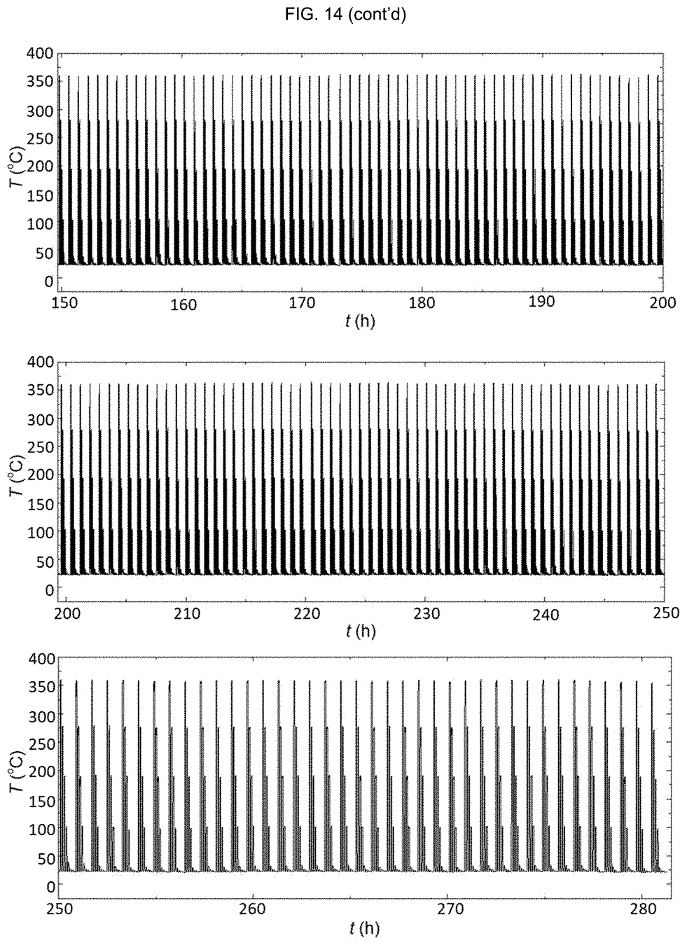

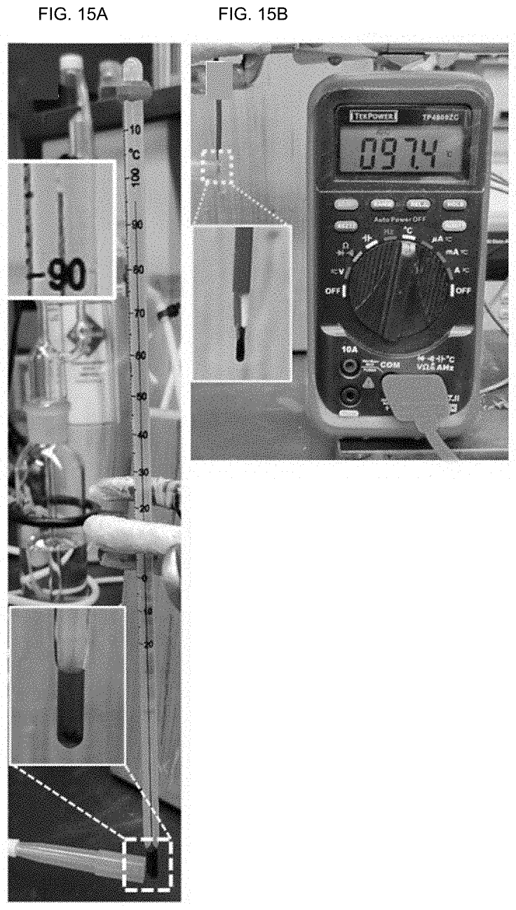

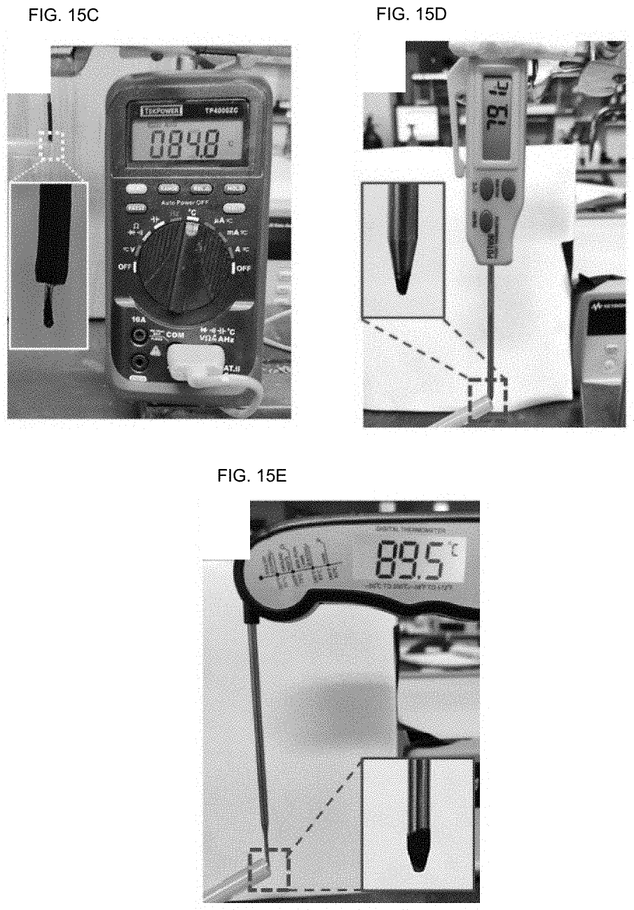

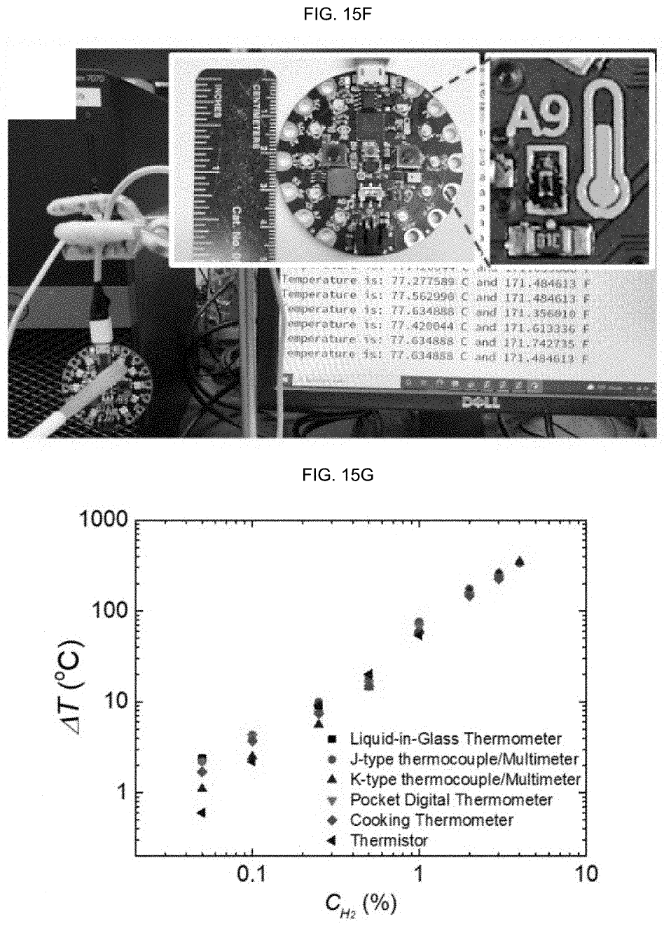

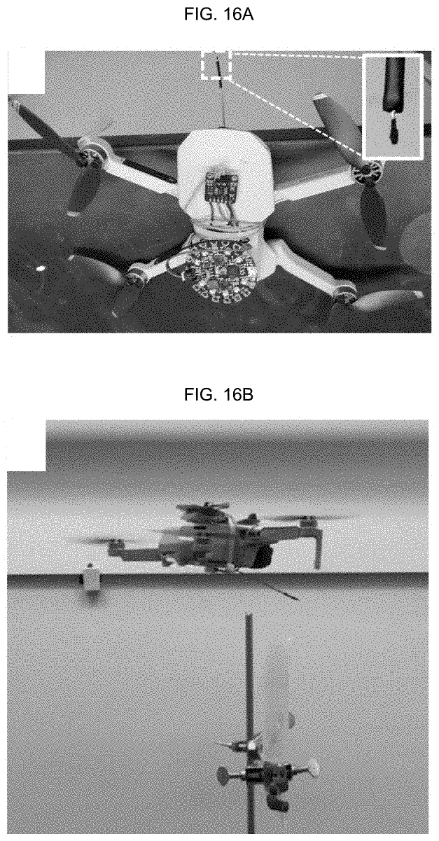

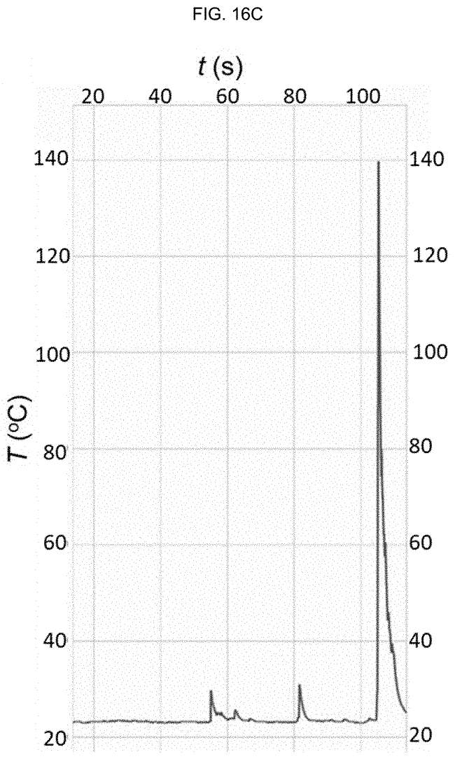

It should be noted that the Pt NP assembly displays low cross-sensitivity (e.g., low sensitivity to compounds other than H₂ reacting in active sites of the Pt NP assembly and providing a false positive H₂ measurement) and anti-poisoning (e.g., low sensitivity to the active sites of the Pt NP assembly being deactivated by compounds in the ambient environment) properties. To evaluate these two metrics accurately, the interferent/cross-sensitivity test protocol developed by the Safety Sensor Testing Laboratory at the DOE National Renewable Energy Laboratory with slight modifications was adopted (FIG. 13). (U.S. Department of Energy, National Renewable Energy Laboratory (NREL), Safety Sensor Testing Laboratory, Standard Hydrogen Test Protocols, Pages 8-9, (2011)) AG. 9F plots the percentage change of sensor temperature when exposed to 36 different interference gases, including CO, NO₂, SO₂, H₂S, NH₃, and various organic gases and vapors such as natural gas, hexane, benzene, formaldehyde, methanol, etc., in the absence and presence of 4% H₂ (see raw data in FIG. 13). It should be noted that the percentage change of sensor temperature was determined relative to an initial state and a response to H₂ that provides a calibrated value. Notably, the interference gases were determined to cause less than a 10% change in the sensor response. In contrast, common commercial catalytic sensors are known to suffer severe cross-sensitivity to combustible gases and vapors (for example, the relative sensitivity for CH₄ and H₂ is 1). (RAE Systems, Inc., RAE Systems Technical Note TN-114, Sensor Specifications and Cross-Sensitivities. Page 5-6) Additionally, and after being exposed to these interference gases, the sensor was determined to return to the initial state and produced the response to H₂ at the calibrated value (FIG. 13), indicating no and/or minimal irreversible poisoning effects from the interference gases. Furthermore, the Pt NP assembly sensor also exhibited high long-term stability. During a 281-hour-long 288-test-cycles test, the sensor response remained substantially constant (FIG. 9G, 14). The relative standard deviation of sensor response was merely 1% for CH₂ 1%. The slight variation in sensor response at a low CH₂ of 0.05% is possibly due to the slight room temperature fluctuation. Because the sensor readout is temperature, H₂ sensors can be fabricated using any commercially available thermometers, including thermocouples, thermistors, and even liquid-in-glass laboratory thermometers (FIG. 15), making the sensor inexpensive (as low as <$5 per unit) and easily miniaturized (size<2 mm). Furthermore, a portable and wireless gas sensing system prototype that can be loaded onto a commercial drone was built and its practicality for H₂ leak detection was demonstrated (FIG. 16).

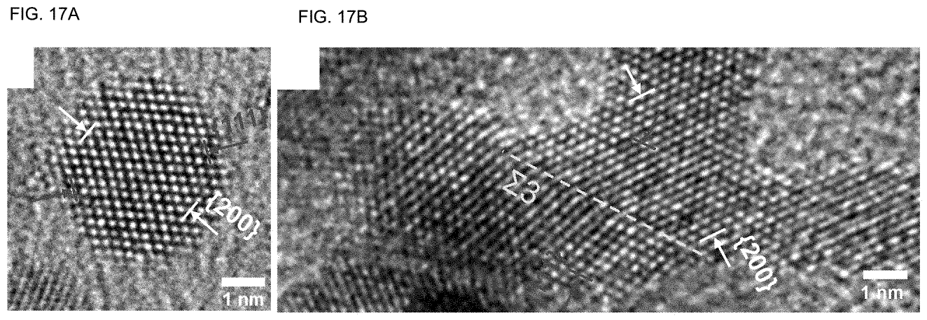

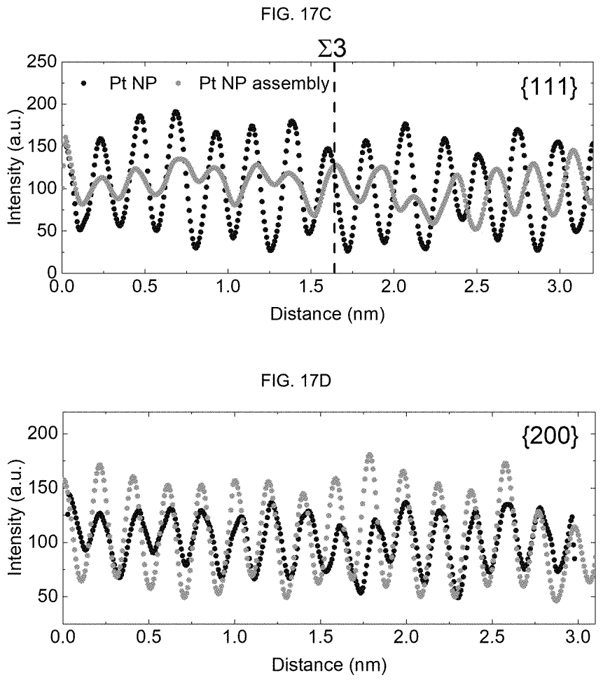

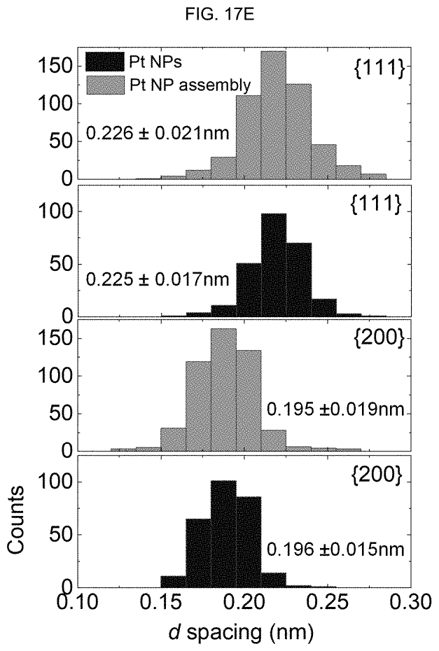

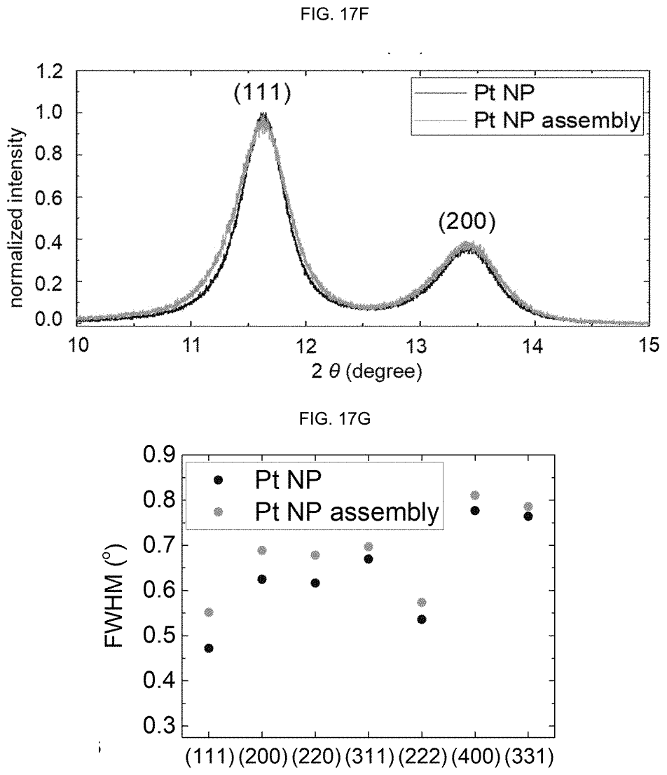

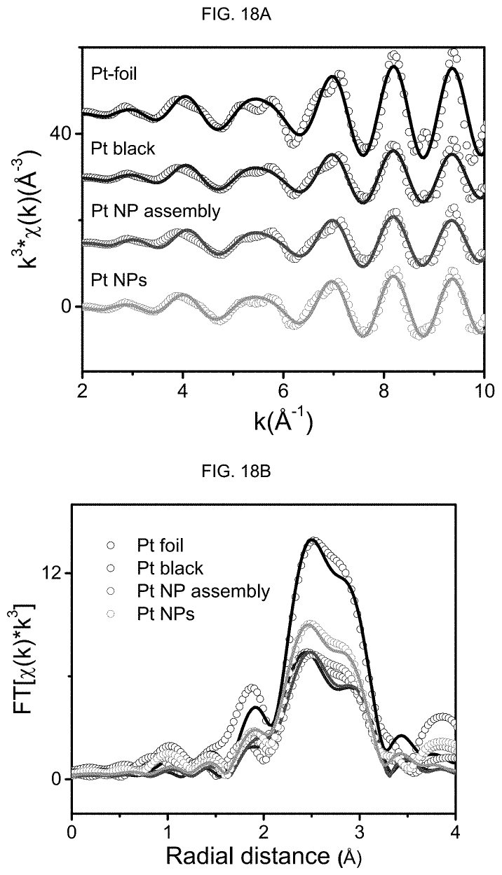

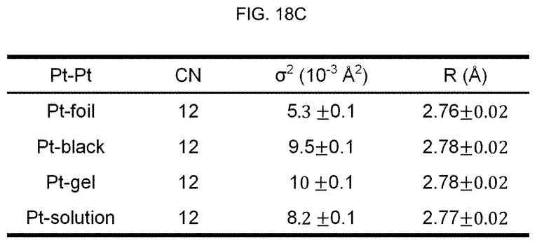

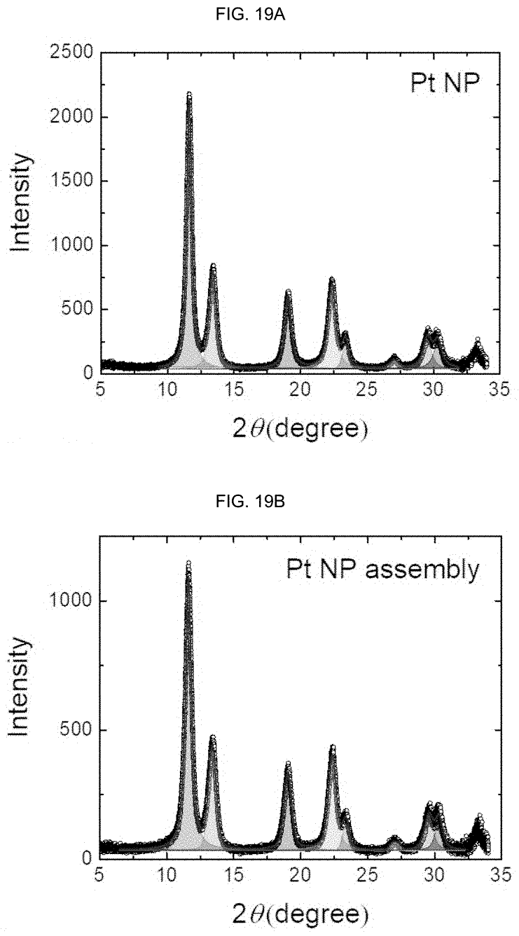

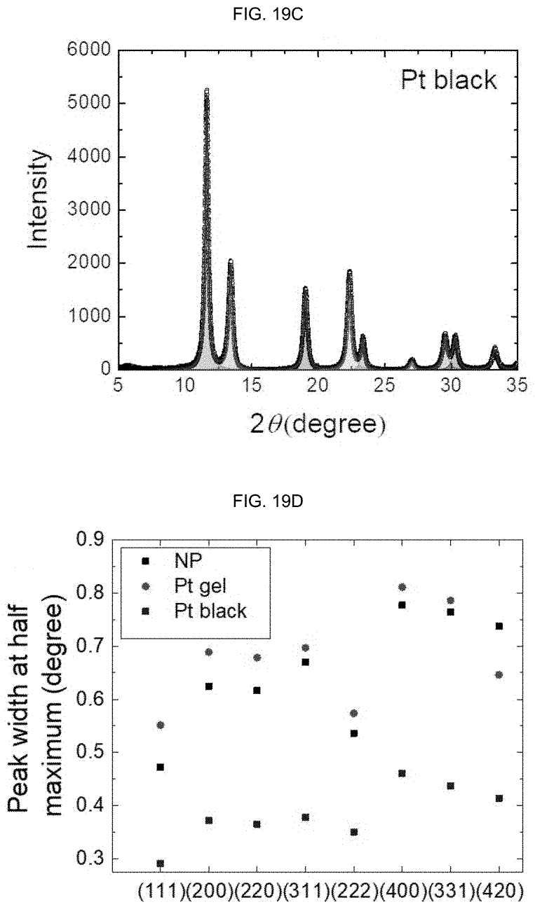

The H₂ sensing performance of the Pt NP assembly is enabled by the Pt NP assembly catalyzing the H₂ oxidation reaction at room temperature. In particular, the Pt NPs display enhanced H₂ oxidation reaction catalyzing the assembly process, when comparing the atomic structures of Pt NPs and the Pt NPs assembly. The {111} and {200} planes were the focus because the representative Σ3 grain boundaries in Pt NP assembly form along the {111} planes (FIG. 17A, 17B). (Grimmer, Bollmann, Warrington, Acta Crystallographica Section A 30, 197-207 (1974)) A lattice expansion of 0.1 nm in the Σ3 grain boundary region of Pt NP assembly was observed, shown by an offset between the TEM intensity profiles along the {111} planes of Pt NP and Pt NP assembly in FIG. 17C. In contrast, there was no offset for their {200} profiles (FIG. 17D). Based on TEM results, statistics of the d-spacing values for {111} and {200} planes revealed that the d-spacing of Pt NP assembly exhibited a broader (20%) distribution than Pt NPs, but their averaged spacings for {111} and {200} are almost the same: 0.226/0.225 nm and 0.195/0.196 nm, respectively (FIG. 17E). Such d-spacing distributions of Pt NPs and Pt NP assembly are in good agreement with their x-ray absorption spectroscopy results: different Debye-Waller factors (Pt NPs: 8.2±0.1 and Pt NP assembly:10±0.1, unit: 10⁻³ Å²) which suggests Pt NP assembly has higher local structural disorder than Pt NPs, but similar fitted Pt—Pt bond lengths (Pt NPs: 0.277±0.002 nm; Pt NP assembly: 0.278±0.002 nm) (FIG. 18). (Vila, et al., Phys. Rev. B 76, 014301 (2007)).

High-resolution x-ray diffraction (XRD) spectra of Pt NPs and Pt NP assembly in FIG. 17F also present similar structural information as the TEM results. The XRD peaks of Pt NP assembly show anisotropic and asymmetric broadening but no peak position change, relative to Pt NPs (FIG. 17F, 17G). Specifically, the (111) peak of Pt NP assembly was broadened by 18% on the left side but 10% on the right at half maximum (FIG. 17F). The peak broadening is more significant for the (111) peak (17%) than others (e.g., 10% for (200) and 4% for (311) in FIG. 17G). Microstrain, crystallite size, and instrumental errors together determine XRD peak broadening but do not change the peak position. (Chattot et al., Nat. Mater. 17, 827-833 (2018); Ungar, Scr. Mater. 51, 777-781 (2004); Venkateswarlu, Chandra Bose, Rameshbabu, Condensed Matter 405, 4256-4261 (2010)) The result in FIG. 17F suggests only increased microstrain was present in the Pt NP assembly, given the same instrumental errors and similar crystallite sizes of Pt NPs and Pt NP assembly (FIG. 1B-1C, 4). The grain boundaries most likely induce the microstrain in Pt NP assembly. The anisotropy and asymmetry in peak broadening are attributed to the dominance of Σ3 grain boundaries that form along the {111} direction in Pt NP assembly and the tensile strain (i.e., lattice expansion in FIG. 17C) associated with these grain boundaries, respectively. (Ungar, Scr. Mater. 51, 777-781 (2004)).

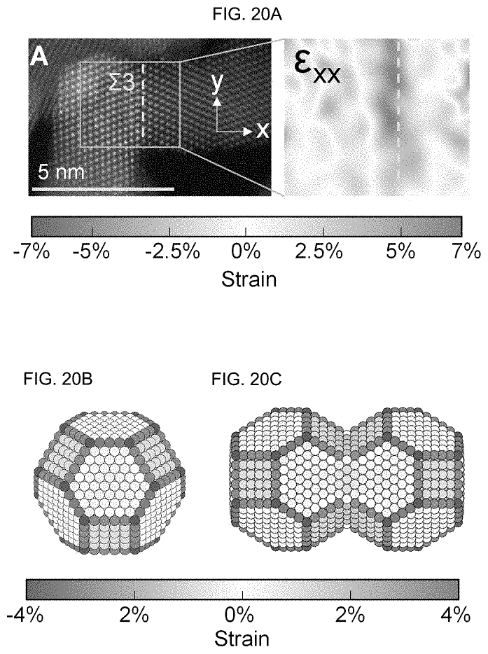

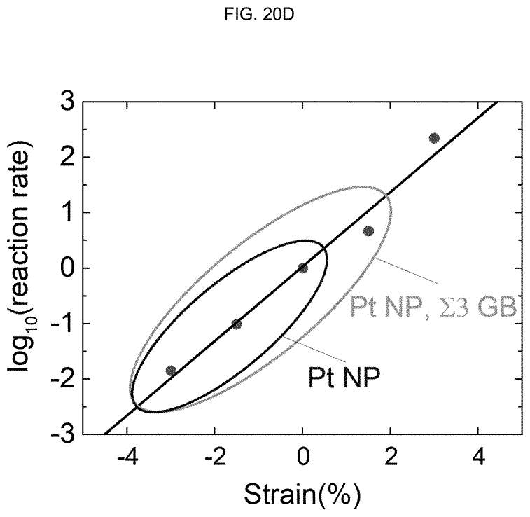

The strain field near grain boundaries in the Pt NP assembly from the atomic-resolution STEM images were further quantitatively analyzed using a geometric phase analysis algorithm. (Hitch, Snoeck, Kilaas, Ultramicroscopy 74, 131-146 (1998)) FIG. 20A shows a tensile strain of up to 7% along the x-axis direction (i.e., the direction normal to {111} planes) is localized at a Σ3 grain boundary. Theoretical simulation results in FIGS. 20B and 20C also confirm the presence of tensile strain at a Σ3 grain boundary formed between two Pt NPs with diameters comparable to experimental ones (3.87 nm). Using strained Pt (111) surface models, an exponential dependence of H₂ oxidation rate on the surface strain was obtained from density functional theory calculations (green dots in AG. 20D). (Qi, Yu, Li, J. Chem. Phys. 125, 054701 (2006)) The drastically accelerated H₂ oxidation rate under a tensile strain is caused by the increased coverage of oxygen species on the catalyst surface. According to the calculated strain values of surface atoms in the Pt NP and NP dimer in FIGS. 20B and 20C, the reaction rate at the grain boundary should be at least 10 times higher than on a Pt NP surface due to this strain effect.

This work demonstrates a facile and scalable approach for synthesizing a grain-boundary-rich Pt NP assembly and its practical use as a high-performance catalytic hydrogen sensor that operates at room temperature. Given the recently discovered high activities of grain boundaries for other catalytic reactions such as CO₂ electroreduction and methane activation (Huang et al., Science 373, 1518-1523 (2021); Mariano, et al., Science 358, 1187-1192 (2017); Li, J. Ciston, M. W. Kanan, Nature 508, 504-507 (2014)) this synthetic approach will be powerful for studying the activity at grain boundaries between NPs because the crystallite size of the NP precursors is largely retained in the NP-assembly, making the comparison between the activities with and without grain boundaries straightforward.

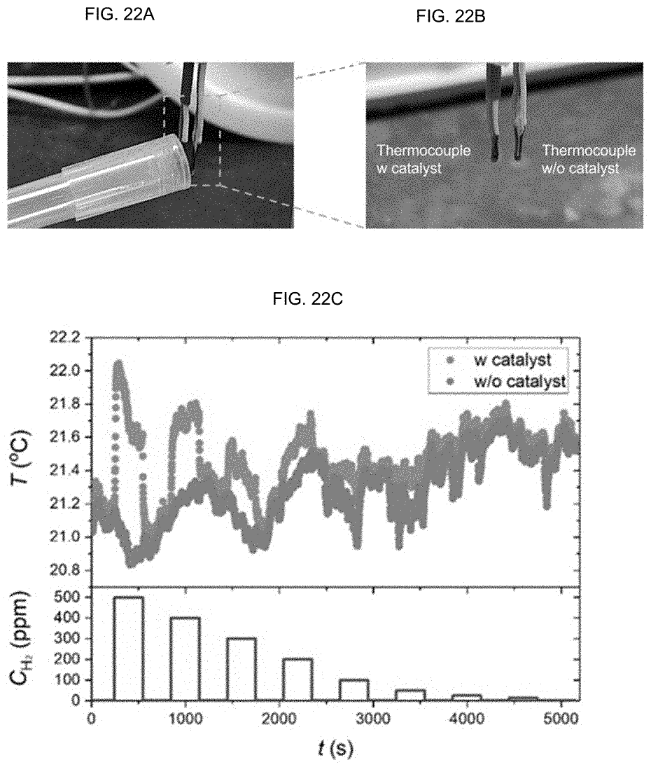

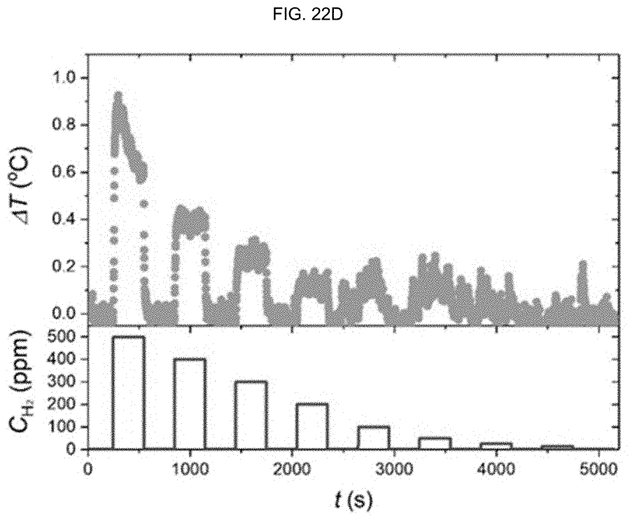

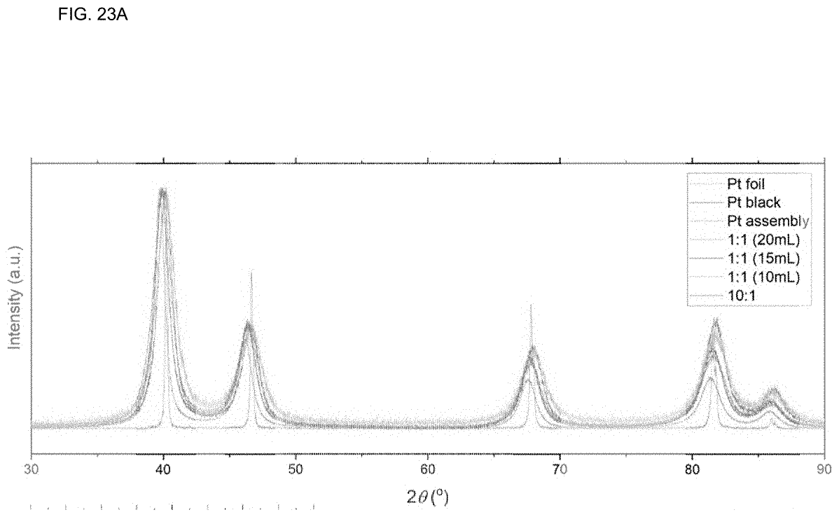

The sensor's limit of detection (LOD) is co-determined by the sensor response and the background noise. The catalytic hydrogen gas sensor uses temperature readout as the sensor response; thus, background noise can arise from ambient temperature fluctuation. The temperature fluctuation in an air-conditioned room is typically 1° C., limiting the sensor's LOD to 500 ppm (FIG. 22C). Under outdoor conditions, environmental temperature fluctuation is more severe, further deteriorating the LOD. To address this limitation, a baseline subtraction strategy was developed. A second thermocouple without any catalyst coating as a reference was added and placed next to the sensing thermocouple to measure the environmental temperature fluctuation (FIG. 22A-22B). After subtracting the baseline signal recorded by the reference thermocouple from the sensor signal, the LOD was lowered to 50 ppm (FIG. 22D).

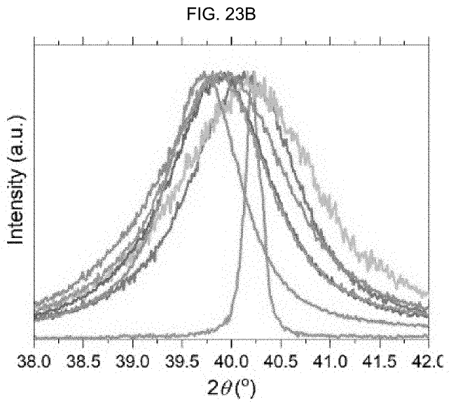

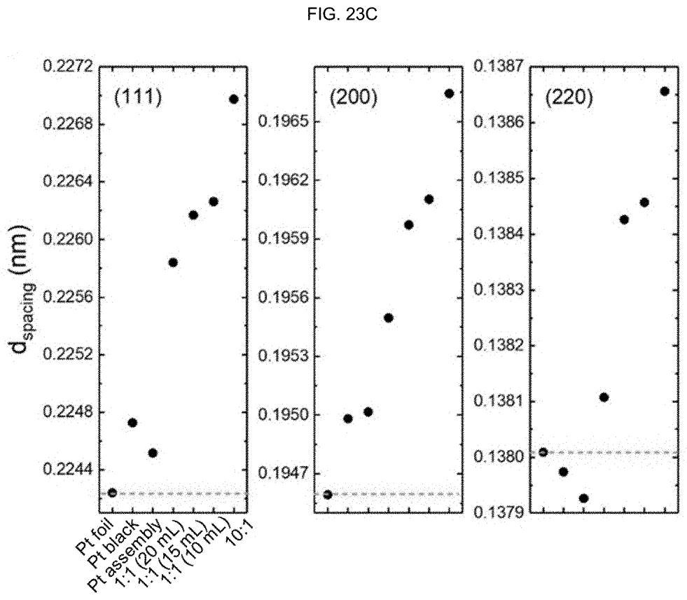

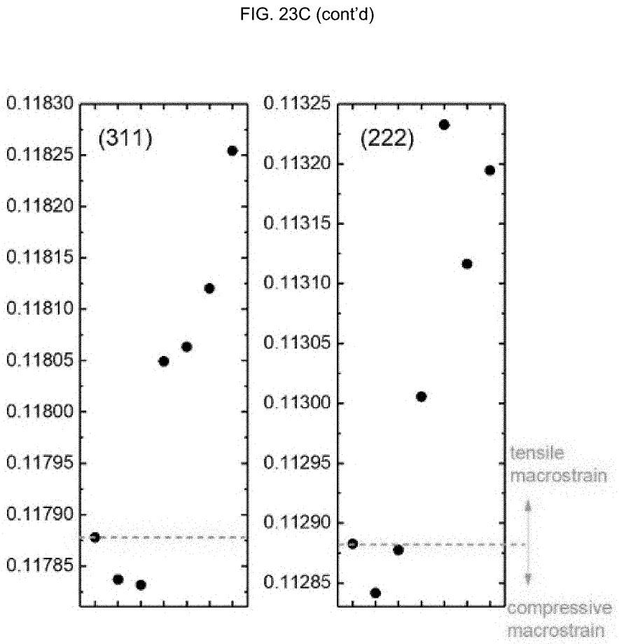

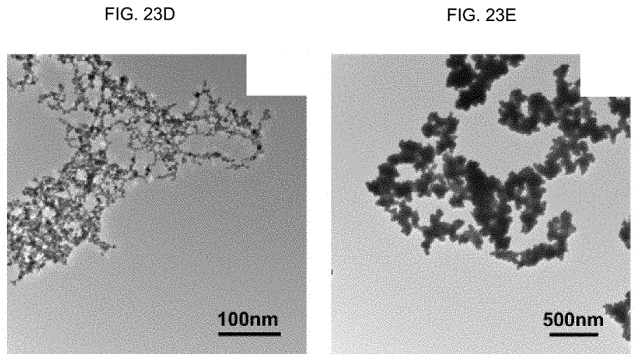

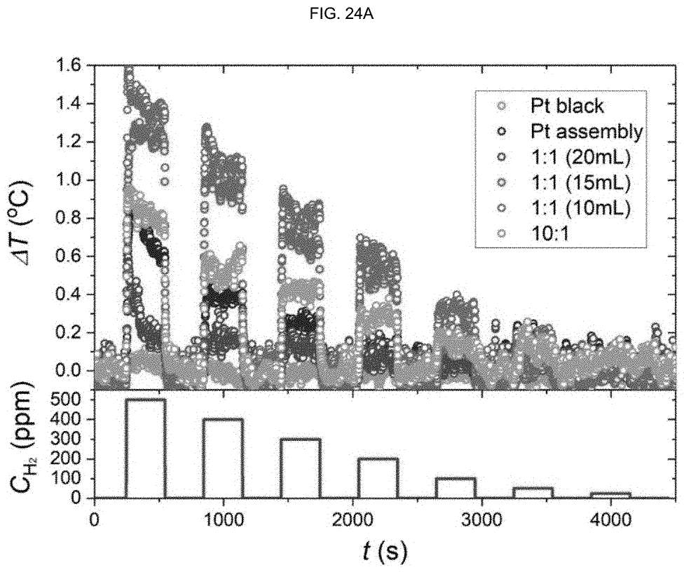

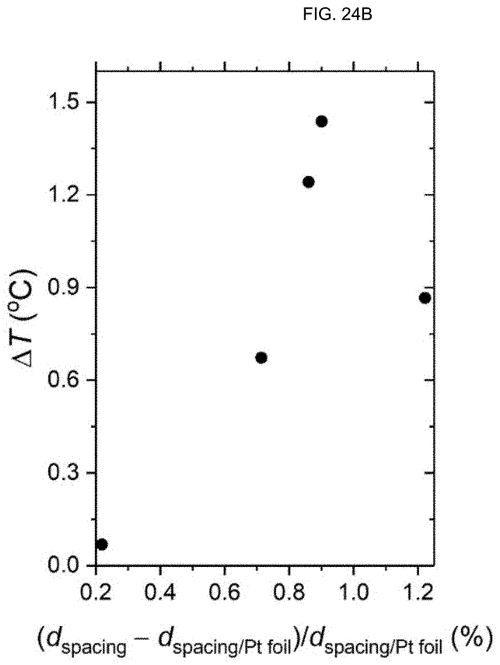

The mechanistic study discussed earlier shows that the tensile strain in Pt catalysts would improve the catalytic hydrogen oxidation activity. Thus, tensile strain engineering is a second strategy to further improve sensing performance. Specifically, Pt materials with tunable tensile strain were synthesized by adding various concentrations of NaBH₄ into a Na₂PtCl₆ aqueous solution. Without adding capping ligands, NaBH₄ would rapidly reduce Pt precursor to Pt atoms/clusters, which attach and aggregate disorderly to induce lattice strain. As NaBH₄ concentration increases (or the added NaBH₄ solution volume decreases), the Pt catalyst morphology changes from a highly porous nanowire structure connected by individual nanoparticles into a dense aggregate (FIGS. 23D-23E) because the high supersaturation level of Pt atoms/clusters at high NaBH₄ concentrations switches from the nanoparticle aggregation from diffusion-limited process to reaction-limited one. An apparent XRD peak shift to the smaller angle side at high NaBH₄ concentrations indicates an increasing tensile strain in the Pt material (FIGS. 23A-23C). The H₂ sensing tests in the ppm range show that the tensile strain dramatically boosts the sensor performance. The response of 0.9% tensile strained Pt (1:1, 10 mL) increased by 114% relative to 0.7% strained one (1:1, 20 mL), and LOD was improved from 200 ppm to 25 ppm (FIG. 24A-24B). Limited by the current condition of diluting H₂ gas, 25 ppm is the lowest concentration that was able to be tested with confidence. Therefore, the true LOD could be lower than 25 ppm.

Experimental Methods. Material synthesis. Preparation of Pt NPs. Pt NPs were synthesized using a previously reported approach with slight modifications (Kenneth, Brown, Natan, Chem. Mater. 2000, 12 (2), 306-313). Specifically, 35 mL aqueous solution of 0.14 mmol hydrogen hexachloroplatinate(IV) hexahydrate (H₂PtCl₆, Sigma Aldrich) was added to 460 mL of DI water at 100° C. One minute later, 11.6 mL aqueous solution of 0.42 mmol sodium citrate tribasic dihydrate (C₆H₉Na₃O₉, Sigma Aldrich) was added. After one minute, 5 mL of the freshly prepared ice-cold aqueous mixed solution of 0.14 mmol sodium borohydride (NaBH₄, Sigma Aldrich) and 0.28 mmol C₆H₉Na₃O₉. After ten minutes, the grayish-brown solution was cooled down to room temperature naturally.

Determining the Pt NP concentration. Before electrochemical NP assembly, the as-synthesized 500 mL Pt NP solution was concentrated by 10 fold to a final volume of 50 mL at 75° C. using a rotary evaporator. The concentration of the concentrated Pt NP solution was estimated to be 1.5 μM as follows:

The volume of a Pt NP with a diameter of 3.6 nm (the average Pt size based on TEM images):

$${V}_{Pt NPs} = \frac{4}{3} π {r}^{3} = 24.4 {nm}^{3}$$

The volume of a face-center cubic (fcc) Pt unit cell is

V_{Pt unit cell}=0.06 nm³ according to the standard PDF card No. 00-004-0802

The number of unit cells in one Pt NP:

N_{cell}=V_{Pt NPs}/V_{Pt unit cell}=407

The total number of Pt atoms in one Pt NP:

N_{Pt}=4×N_{cell}=1628

The concentration of Pt atoms in the concentrated Pt NP solution:

C_{Pt}=n_{Pt}/V_{concentrated}=2.8mM

The Pt NP concentration:

C_{Pt NPs}=C_{Pt}/N_{Pt}=1.7μM

Electrochemical synthesis of wet Pt NP assemblies. A three-electrode system was utilized to synthesize Pt NP assemblies by linking Pt NPs into chain-like Pt NP networks. A Pt foil (240 mm²), a Pt foil (385 mm²), and an Ag/AgCl electrode in saturated KCl aqueous solution were used as the working electrode, the counter electrode, and the reference electrode, respectively. The term “saturated” refers to being at a point at which a solution of a substance can dissolve no more of that substance. Prior to electrochemical synthesis, two Pt foils were electrochemically polished in 0.5 M H₂SO₄ aqueous solution by cycling the electrode potential between 1.1 V and −0.23 V at a sweep rate of 0.1 V/s for 500 cycles using a CHI 650E potentiostat. The Ag/AgCl reference electrode and two Pt foils were rinsed using DI water several times. In a typical electrochemical synthesis experiment, 8 mL of concentrated Pt NP solution was added to a 10 mL glass beaker. A constant electrode potential of −2 V was applied at the working electrode for five hours until the supernatant color turned transparent, indicating the completion of NP assembly. Finally, the supernatant was carefully removed using a pipette, and the wet Pt NP assemblies at the bottom were washed with DI water ten times in one week.

Preparation of dry Pt NP assemblies. Critical Point Drying (CPD) was employed to prepare a dry Pt NP assembly powder from wet Pt NP assemblies. Before CPD, the solvent in wet Pt NP assemblies was exchanged from DI water to acetone by removing the supernatant carefully, and then the same amount of acetone was added. This solvent exchange step was repeated three times a day for one week. Next, the wet Pt NP assemblies were supercritically dried using an SPI-DRY model CO₂ critical point dryer with a recirculating water bath (ISOTEMP 10065). The acetone was first completely replaced with liquid CO₂ at 18° C., and liquid CO₂ was evaporated by increasing temperature to 37° C. Thirty minutes later, the CPD process was completed, and dry Pt NP assemblies were obtained.

Characterizations. High-angle annular dark-field scanning transmission electron microscopy (HAADF-STEM) images were taken using JEOL 3100R05 TEM (Double Cs Corrected TEM/STEM) and Thero Fisher Titan (probe Cs Corrected TEM, 300 KV). HRTEM images were collected using Thermo Fisher Titan (imaging Cs Corrected TEM, 300 KV). The particle size distribution was estimated using a Nano Measurer 1.2 software. The crystal structure was measured by Powder X-ray diffraction (PXRD) using a Bruker D2 Phaser diffractometer. PXRD patterns were analyzed by the powder diffraction file database of the International Center for Diffraction Data. The chemical state and elementary ratio were analyzed by X-ray Photoelectron Spectroscopy (XPS, ThermoFisher Scientific NEXSA UV and X-ray Photoelectron Spectrometer). XPS peaks were fitted using a composite function (30% Lorentzian+70% Gaussian) and calibrated according to the C 1s peak at 284.8 eV via the Avantage software. Steady-state X-ray absorption spectroscopy (XAS) measurement was performed at 12-BM beamline at the Advanced Photon Source (APS), Argonne National Laboratory. The XAS data were collected under room temperature with fluorescence mode using a 13-element germanium solid-state detector. Three ion chambers were used, where one of them was placed before the sample for the incident X-ray flux reference signal. The other two ion chambers (second and third chambers) are placed after the sample. The Pt foil is placed between the second and third ion chambers and used for collecting Pt metal XAS spectrum for energy calibration.

H₂ Sensor fabrication. Six commercially available thermometers, including a J-type thermocouple wire (NEW Keysight Agilent, Part number: 34970-61606, Measuring range: −40° C.-750° C.), a K-type thermocouple wire (Adafruit, Part number: 270, Measuring range: −40° C.-500° C.), a liquid-in-glass thermometer (SP Bel-Art, Part number: 13-201-663, Measuring range: −20° C.-110° C.), a cooking thermometer (Pilita, Part number: DT-68, Measuring range: −50° C.-300° C.), a digital pocket thermometer (UEi Test Instruments, Part number: PDT650, Measuring range: −50° C.-300° C.) and a thermistor (Adafruit, Part number: 3915, Measuring range: −20° C.-120° C.), were used to prepare a catalytic H₂ sensor. A wet slurry containing 1 mg Pt NP assembly (prepared by mixing 1 mg Pt NP assembly with 100 μL ethanol) was drop-casted on the sensing region of a thermometer and dried in air to fabricate a corresponding H₂ sensor.

In control experiments using other Pt catalysts, 1 mg of Pt NPs (precipitated by centrifuge), Pt NPs without ligand (precipitated by adding NaOH aqueous solution with pH=14), Pt black (Sigma Aldrich), Pt powder (Sigma Aldrich), and Adams' catalyst (Sigma Aldrich) were dispersed in 100 μL ethanol to prepare their corresponding slurries. The obtained slurries were drop-casted on the tip of a J-type thermocouple wire and then dried to prepare the catalytic H₂ sensors.

Gas sensing tests. The standard H₂ sensing tests in this study were carried out using sensors prepared from a J-type thermocouple wire (abbreviated as J-H₂ sensors) unless otherwise specified.

Standard H₂ sensing test protocol. The H₂ sensing tests were carried out using a home-built apparatus at room temperature, which includes two gas cylinders (synthetic air and H₂ from Airgas Co., Ltd) and mass flow controllers, a data acquisition meter (Keysight/Agilent 34972A LXI) for real-time recording of the temperature readout, and PC Computer (Hewlett-Packard Co., Ltd) for storing data (Geng et al., Nat Commun 2021, 12 (1), 4895). A J-H₂ sensor was placed directly in front of the gas outlet during the test (FIG. 9A insert). Before exposure to H₂, the J-H₂ sensor was first stabilized in synthetic air (21% O₂+79% N₂) at a flow rate of 1000 sccm regulated by a mass flow controller at room temperature and relative humidity of 0%. Then, H₂ gas was mixed with synthetic air to achieve various H₂ concentrations of 4%, 3%, 2%, 1%, 0.5%, 0.25%, 0.1%, and 0.05%. These H₂/air mixtures sequentially flowed over the J-H₂ sensor at a constant rate of 1000 sccm to obtain the response/recovery curves of J-H₂ sensors.

Illustrative Examples. In some examples, metallic NPs can be provided to an electrolysis reactor via a feed solution that contains NPs suspended within a solvent and stabilized by a ligand compound. The electrolysis reactor can be activated to provide a voltage via a first electrode and a second electrode of the electrolysis reactor to the feed solution. By monitoring the state of the feed solution, the formation of a nanoparticle assembly can be monitored to determine whether the NPs have been assembled to form a nanoparticle assembly. As noted above, the nanoparticle assembly includes the NPs connected via one or more grain-boundaries. The feed solution can be provided to the electrolysis reactor in a first state that is characterized by at least one of a first color or a first opacity. Similarly, formation of the nanoparticle assembly can be characterized by the feed solution transitioning to a second state characterized by at least one of a second color or a second opacity. The electrolysis reactor can be activated at first time to initiate formation of the nanoparticle assembly(s) and deactivated at a second time based on a determination that the nanoparticle assembly(s) have been formed. Accordingly, the nanoparticle assembly(s) can be extracted from the solvent of the feed solution and purified to remove remnants of the feed solution, the NPs that were not incorporated into the nanoparticle assembly(s), and remnants of the ligand compound.

It should be noted that the NPs can be formed from a platinum ion precursor that is provided to the solvent (e.g., hydrogen hexachloroplatinate(IV) hexahydrate, hexachloroplatinate, platinous chloride, etc.). Additionally, a ligand precursor can be provided to the solvent (e.g., sodium polyacrylic acid, sodium citrate, etc.) to act as a capping agent that stabilizes the Pt NPs and prevents aggregation/coalescence of the Pt NPs. Further, a reducing agent can be provided to the solvent (e.g., hydrogen gas, sodium borohydride, ethylene glycol, etc.), the reducing agent causing the Pt NPs to form within solvent and the ligand precursor to combine with the Pt NPs as the ligand compound. When exposed to the voltage of the electrolysis reactor, the ligand compound can disassociate from the Pt NPs. Additionally, the gas bubbles generated from the solvent agitate the feed solution. Due to the agitation from the gas bubbles and destabilizing the nanoparticles via removal of the ligand compound, the nanoparticle assembly can form as the individual nanoparticles combine and attach at grain-boundaries. Accordingly, the nanoparticle assembly(s) can include the Pt NPs in the feed solution combined via grain-boundaries. As noted above, grain-boundaries can be transition regions that exist between crystalline structures of the Pt NPs combined to form the nanoparticle assembly.

In some examples, the one or more grain-boundaries include different types of grain-boundaries dependent on how the individual NPs combined. For example, a highly-disordered grain-boundary can exist where a first crystalline structure meets a second crystalline structure and is offset from the second crystalline structure by a first angle greater than 15 degrees. Similarly, a low-disorder grain-boundary can exist where a third crystalline structure meets a fourth crystalline structure and is offset from the fourth crystalline structure by a second angle less than 15 degrees. Further, the crystalline structures within the nanoparticle assembly can have an average structure diameter that is substantially equal to the average diameter of the NPs within the feed solution. Within this context, and in particular embodiments, “substantially equal” means within 1 nm in size, within 0.5 nm in size, or within 0.3 nm in size.

Hydrogen sensors that are formed through application of the nanoparticle assembly to a temperature sensor can monitor hydrogen utilizing processes to detect leaks that present a potential safety hazard. For example, the hydrogen sensor can be associated with hydrogen sources that are configured to provide hydrogen to a larger system, fluid valves that regulate the flow of hydrogen within processes, hydrogen lines that carry hydrogen between individual systems, and other such applications that consume or utilize hydrogen. Additionally, the hydrogen sensor can be associated with a hydrogen sensor controller that is configured to monitor the hydrogen sensor and determine when the hydrogen sensor is detecting hydrogen concentrations that exceed a concentration threshold or other threshold value. More specifically, the hydrogen sensors can be utilized, via the hydrogen sensor controller or other controller to determine whether hydrogen is leaking within a facility and enable the hydrogen leak to be identified and sealed to prevent ignition/combustion events fed by the leaking hydrogen. Accordingly, the hydrogen sensor can be mounted on or otherwise associated with hydrogen systems to identify hydrogen leaks.

In some examples, and as noted elsewhere herein, the hydrogen sensor can include a temperature sensor that is associated with the hydrogen sensor controller and a nanoparticle assembly coating that is applied to the temperature sensor. The nanoparticle assembly coating is made from the nanoparticle assembly being deposited (e.g., via drop-casting) and generates thermal energy when exposed to hydrogen. The temperature sensor can transmit an indication of temperature associated with the nanoparticle assembly coating, wherein the nanoparticle assembly coating generates thermal energy and causes elevated temperatures to be detected via the temperature sensor when hydrogen contacts the nanoparticle assembly coating. The hydrogen oxidation reaction catalyzed by the nanoparticle assembly is an exothermic reaction that generates increased amounts of thermal energy in the presence of increased amounts of hydrogen. Accordingly, the hydrogen sensor controller can determine when the hydrogen sensor detects hydrogen exceeding a lower flammable limit of the hydrogen in air. In particular, the hydrogen sensor controller can correlate the temperature detected by the temperature sensor and the concentration of hydrogen oxidized by the nanoparticle assembly to cause the temperature. Thus, a warning can be activated by the hydrogen sensor controller and the hydrogen sensor when the amount and/or the concentration of hydrogen exceeds the lower flammable limit and poses a combustion hazard.

In some examples, the hydrogen sensor can be created by the metallic NP assembly being deposited onto a temperature sensor (e.g., a J-type thermocouple, a K-type thermocouple, a thermistor, etc.). In particular, the NP assembly can be deposited onto the temperature sensor via drop-casting to form a thin film of the NP assembly on the temperature sensor. Alternatively, or in addition, the NP assembly can be applied directed to the temperature sensor through spray coating, electrophoretic processes, ultrasonic dispersion, and other related deposition processes. More specifically, the nanoparticle assembly can be extracted and purified from the electrolysis reactor after formation of the nanoparticle assembly to obtain a nanoparticle assembly powder. The nanoparticle assembly powder can be mixed with an alcohol or other solvent to form a nanoparticle slurry. The temperature sensor can be submerged in the nanoparticle slurry to apply a thin film of the nanoparticle assembly to the temperature sensor and form a hydrogen sensor. It should be noted that the solvent can be selected to wet the surface of the temperature sensor and deposit the nanoparticle assembly on the temperature sensor.

### Exemplary Embodiments

1. A method including:
receiving, within a receptacle of an electrolysis reactor, a solvent that contains platinum nanoparticles that are suspended within the solvent by a ligand compound;
activating, at a first time, a first electrode and a second electrode of the electrolysis reactor to apply a voltage to the solvent; and
determining, based at least on a state of the solvent, that platinum nanoparticles within the platinum nanoparticles have been assembled to form a nanoparticle assembly, the platinum nanoparticles of the nanoparticle assembly connected via one or more grain-boundaries.
2. The method of embodiment 1, wherein:
the first electrode is formed from a first platinum foil, the first electrode configured as a positive electrode for providing the voltage;
the second electrode is formed from a second platinum foil, the second electrode configured as a negative electrode for providing the voltage; and
wherein the electrolysis reactor utilizes a third electrode that is formed from silver and silver chloride, the third electrode configured as a reference electrode for the voltage.
3. The method of embodiment 1 or 2, wherein the receptacle further includes a base solution that the solvent, the platinum nanoparticles, and the ligand compound are added to.
4. The method of embodiment 3, wherein the base solution is an aqueous solution that is saturated with potassium chloride and the first electrode, the second electrode, and the third electrode are at least partially submerged in the base solution.
5. The method of any of embodiments 1-4, wherein receiving the platinum nanoparticles further includes:
receiving a platinum ion precursor that is provided to the solvent;
receiving a ligand precursor that is provided to the solvent; and
receiving a reducing agent that is provided to the solvent, the reducing agent causing the platinum nanoparticles to form within the solvent and the ligand precursor to combine with the platinum nanoparticles as the ligand compound.
6. The method of embodiment 5, wherein:
the platinum ion precursor is hydrogen hexachloroplatinate(IV) hexahydrate;
the ligand precursor is sodium citrate tribasic dihydrate; and
the reducing agent is sodium borohydride.
7. The method of any of embodiments 1-6, wherein the ligand compound is a capping agent that reduces or prevents individual nanoparticles of the platinum nanoparticles from combining with each other within the solvent.
8. The method of any of embodiments 1-7, wherein:
the voltage is determined to remove the ligand compound from the platinum nanoparticles and destabilize the platinum nanoparticles; and
the voltage causes the platinum nanoparticles to collide and combine to form the nanoparticle assembly after removal of the ligand compound.
9. The method of embodiment 8, wherein the voltage electrolyzes the solvent such that one or more gaseous compounds are output by the solvent, the one or more gaseous compounds forming bubbles that agitate the platinum nanoparticles and cause collisions between the platinum nanoparticles.
10. The method of any of embodiments 1-9, wherein determining that the platinum nanoparticles have been assembled into the nanoparticle assembly includes:
determining a first state of the solvent, the first state characterized by at least one of a first color or a first opacity;
determining that the solvent has transitioned from the first state to a second state, the second state characterized by at least one of a second color or a second opacity and the solvent transitions from the first state to the second state due to the platinum nanoparticles forming the nanoparticle assembly.
11. The method of any of embodiments 1-10, wherein the nanoparticle assembly includes the platinum nanoparticles of the amount that are combined via the one or more grain-boundaries, the one or more grain-boundaries being transition regions between a subset of the platinum nanoparticles that form the nanoparticle assembly.
12. The method of embodiment 11, wherein the one or more grain-boundaries include:
a highly-disordered grain-boundary where a first crystalline structure meets a second crystalline structure and is offset from the second crystalline structure by a first angle greater than 15 degrees; and
a low-disorder grain-boundary where a third crystalline structure meets a fourth crystalline structure and is offset from the fourth crystalline structure by a second angle less than 15 degrees.
13. The method of embodiment 11, wherein:
the platinum nanoparticles have a first diameter within the solvent; and
the platinum nanoparticles of the nanoparticle assembly have a second diameter, the first diameter being substantially equal to the second diameter.
14. The method of any of embodiments 1-13, further including extracting the nanoparticle assembly from the solvent, wherein extracting the nanoparticle assembly includes at least one of: removing the solvent from the nanoparticle assembly via pipette;
filtering the nanoparticle assembly from the solvent; or
evaporating the solvent.
15. The method of any of embodiments 1-14, further including cleaning the nanoparticle assembly to remove remnants of the solvent, additional platinum nanoparticles that are separate from the nanoparticle assembly, and remnants of the ligand compound.
16. The method of embodiment 15, wherein cleaning the nanoparticle assembly includes repeated rinsing of the nanoparticle assembly with a second solvent.
17. The method of any of embodiments 1-16, further including drying the nanoparticle assembly, wherein drying the nanoparticle assembly includes:
submerging the nanoparticle assembly in a second solvent;
providing the nanoparticle assembly within the second solvent to a critical point drier; exchanging the second solvent with a third solvent at a first temperature; and evaporating the third solvent at a third temperature to obtain the nanoparticle assembly.
18. A system including:
a hydrogen source that is configured to provide hydrogen via a fluid valve and a hydrogen line; a hydrogen sensor controller that is associated with the hydrogen source and configured to determine whether at least one of the hydrogen source is leaking an amount of the hydrogen exceeding a threshold value;
a hydrogen sensor that is associated with hydrogen source and the fluid valve, the hydrogen sensor including:
a temperature sensor that is associated with the hydrogen source and the hydrogen sensor controller; and
a nanoparticle assembly coating that is applied to the temperature sensor, the nanoparticle assembly coating including a nanoparticle assembly generates thermal energy when exposed to the hydrogen from the hydrogen source; and
wherein, the hydrogen sensor transmits an indication of temperature, generated by the temperature sensor from the thermal energy generated by the nanoparticle assembly coating, to the hydrogen sensor controller, the indication causing the hydrogen sensor controller to determine the amount of the hydrogen that is leaking and whether the amount of the hydrogen exceeds the threshold value.
19. The system of embodiment 18, wherein the threshold value is associated with a lower flammable limit of the hydrogen in air.
20. The system of embodiment 18 or 19, wherein the hydrogen sensor is configured to activate a warning in response to determining that the amount of the hydrogen leaking from the hydrogen source exceeds the threshold value.
21. The system of any of embodiments 18-20, wherein the nanoparticle assembly coating catalyzes an oxidation reaction of the hydrogen, the oxidation reaction being exothermic and generating the thermal energy.
22. The system of embodiment 21, wherein the thermal energy generated by the nanoparticle assembly coating is correlated with the amount of the hydrogen that is leaking from the hydrogen source.
23. A method including:
receiving, within an electrolysis reactor, a solvent that contains platinum nanoparticles that are suspended within a first solvent and are stabilized by a ligand compound;
activating, at a first time, a first electrode and a second electrode of the electrolysis reactor to apply a voltage to the solvent;
determining, based at least on the solvent, that platinum nanoparticles of the amount have been assembled to form a nanoparticle assembly, the platinum nanoparticles of the nanoparticle assembly connected via one or more grain-boundaries;
extracting the nanoparticle assembly from the solvent, the nanoparticle assembly being purified to obtain a nanoparticle assembly powder;
mixing the nanoparticle assembly powder with a second solvent to form a nanoparticle slurry; forming a hydrogen sensor from a temperature sensor and the nanoparticle slurry, wherein the temperature sensor is coated with the nanoparticle assembly to create the hydrogen sensor.
24. The method of embodiment 23, wherein the second solvent is an alcohol that the nanoparticle assembly powder is dissolved in.
25. The method of embodiment 23 or 24, wherein the nanoparticle slurry is a suspension of the nanoparticle assembly within the second solvent.
26. The method of any of embodiments 23-25, wherein forming the hydrogen sensor further includes submerging the temperature sensor within the nanoparticle slurry to coat the temperature sensor with the nanoparticle assembly.
27. The method of embodiment 26, wherein the second solvent is evaporated from the nanoparticle slurry that coats the temperature sensor to deposit the nanoparticle assembly on the temperature sensor.
28. A nanoparticle assembly including:
a plurality of platinum nanoparticles that have coalesced to form the nanoparticle assembly and are connected by one or more nanoparticle interfaces;
one or more grain boundaries that form at a subset of the one or more nanoparticle interfaces, the one or more grain boundaries being a transition region between a first crystalline structure of a first platinum nanoparticle and a second crystalline structure of a second nanoparticle; and
a plurality of catalyst active sites that catalyze an oxidation reaction for hydrogen, wherein the one or more grain boundaries enable hydrogen to interact with the plurality of catalyst active sites and undergo the oxidation reaction.

In certain examples, a nanoparticle assembly is grain-boundary rich if at least 10% of nanoparticles within the assembly share a grain boundary with an adjacent nanoparticle. In certain examples, a nanoparticle assembly is grain-boundary rich if at least 20% of nanoparticles within the assembly share a grain boundary with an adjacent nanoparticle. In certain examples, a nanoparticle assembly is grain-boundary rich if at least 30% of nanoparticles within the assembly share a grain boundary with an adjacent nanoparticle. In certain examples, a nanoparticle assembly is grain-boundary rich if at least 40% of nanoparticles within the assembly share a grain boundary with an adjacent nanoparticle. In certain examples, a nanoparticle assembly is grain-boundary rich if a majority of nanoparticles within the assembly share a grain boundary with an adjacent nanoparticle. In certain examples, a nanoparticle assembly is grain-boundary rich if at least 60% of nanoparticles within the assembly share a grain boundary with an adjacent nanoparticle. In certain examples, a nanoparticle assembly is grain-boundary rich if at least 70% of nanoparticles within the assembly share a grain boundary with an adjacent nanoparticle. In certain examples, a nanoparticle assembly is grain-boundary rich if at least 80% of nanoparticles within the assembly share a grain boundary with an adjacent nanoparticle. In certain examples, a nanoparticle assembly is grain-boundary rich if at least 90% of nanoparticles within the assembly share a grain boundary with an adjacent nanoparticle.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art.

1. A method comprising:
receiving, within a receptacle of an electrolysis reactor, a solvent that contains platinum nanoparticles suspended within the solvent by a ligand compound;
activating a first electrode and a second electrode of the electrolysis reactor to apply a voltage to the solvent; and
determining, based at least on a state of the solvent, that individual platinum nanoparticles have assembled into a nanoparticle assembly, the platinum nanoparticles of the nanoparticle assembly connected via one or more grain-boundaries.

2. The method of claim 1, wherein:
the first electrode is formed from a first platinum foil and is a positive electrode for providing the voltage;
the second electrode is formed from a second platinum foil and is a negative electrode for providing the voltage; and
wherein the electrolysis reactor utilizes a third electrode formed from silver and silver chloride, the third electrode configured as a reference electrode for the voltage.

3. The method of claim 2, wherein the receptacle further comprises a base solution that the solvent, the platinum nanoparticles, and the ligand compound are added to.

4. The method of claim 3, wherein the base solution is an aqueous solution that is saturated with potassium chloride and the first electrode, the second electrode, and the third electrode are at least partially submerged in the base solution.

5. The method of claim 1, wherein receiving the platinum nanoparticles further comprises:
receiving a platinum ion precursor that is provided to the solvent;
receiving a ligand precursor that is provided to the solvent; and
receiving a reducing agent that is provided to the solvent, the reducing agent causing the platinum nanoparticles to form within the solvent and the ligand precursor to combine with the platinum nanoparticles as the ligand compound.

6. (canceled)

7. The method of claim 1, wherein the ligand compound is a capping agent reducing the occurrence of individual nanoparticles of the platinum nanoparticles from combining with each other within the solvent.

8. The method of claim 1, wherein:
the voltage is determined to remove the ligand compound from the platinum nanoparticles and destabilize the platinum nanoparticles; and
the voltage causes the platinum nanoparticles to collide and combine to form the nanoparticle assembly after removal of the ligand compound.

9. (canceled)

10. The method of claim 1, wherein determining that the platinum nanoparticles have been assembled into the nanoparticle assembly comprises:
determining a first state of the solvent, the first state characterized by at least one of a first color or a first opacity;
determining that the solvent has transitioned from the first state to a second state, the second state characterized by at least one of a second color or a second opacity and the solvent transitions from the first state to the second state due to the platinum nanoparticles forming the nanoparticle assembly.

11. The method of claim 1, wherein the nanoparticle assembly comprises the platinum nanoparticles that are combined via the one or more grain-boundaries.

12. (canceled)

13. (canceled)

14. The method of claim 1, further comprising extracting the nanoparticle assembly from the solvent, cleaning the nanoparticle assembly, or drying the nanoparticle assembly.

15-20. (canceled)

21. A system comprising:
a hydrogen source that provides hydrogen via a fluid valve and a hydrogen line;
a hydrogen sensor controller that is associated with the hydrogen source and determines whether at least one of the hydrogen source is leaking an amount of the hydrogen exceeding a threshold value;
a hydrogen sensor that is associated with hydrogen source and the fluid valve, the hydrogen sensor comprising:
a temperature sensor that is associated with the hydrogen source and the hydrogen sensor controller; and
a nanoparticle assembly coating that is applied to the temperature sensor, the nanoparticle assembly coating comprised of a nanoparticle assembly that generates thermal energy when exposed to the hydrogen from the hydrogen source; and
wherein, the hydrogen sensor transmits an indication of temperature, generated by the temperature sensor from the thermal energy generated by the nanoparticle assembly coating, to the hydrogen sensor controller, the indication causing the hydrogen sensor controller to determine the amount of the hydrogen that is leaking and whether the amount of the hydrogen exceeds the threshold value.

22. The system of claim 21, wherein the threshold value is associated with a lower flammable limit of the hydrogen in air.

23. The system of claim 21, wherein the hydrogen sensor activates a warning in response to determining that the amount of the hydrogen leaking from the hydrogen source exceeds the threshold value.

24. The system of claim 21, wherein the nanoparticle assembly coating catalyzes an oxidation reaction of the hydrogen, the oxidation reaction being exothermic and generating the thermal energy.

25. (canceled)

26. The system of claim 21, further comprising a second temperature sensor, wherein the second temperature sensor has no nanoparticle assembly coating.

27. A method comprising:
receiving, within an electrolysis reactor, a solvent that contains platinum nanoparticles that are suspended within a first solvent and are stabilized by a ligand compound;
activating, at a first time, a first electrode and a second electrode of the electrolysis reactor to apply a voltage to the solvent;
determining, based at least on the solvent, that platinum nanoparticles of the amount have been assembled to form a nanoparticle assembly, the platinum nanoparticles of the nanoparticle assembly connected via one or more grain-boundaries;
extracting the nanoparticle assembly from the solvent, the nanoparticle assembly being purified to obtain a nanoparticle assembly powder;
mixing the nanoparticle assembly powder with a second solvent to form a nanoparticle slurry;
forming a hydrogen sensor from a temperature sensor and the nanoparticle slurry, wherein the temperature sensor is coated with the nanoparticle assembly to create the hydrogen sensor.

28. The method of claim 27, wherein the second solvent is an alcohol that the nanoparticle assembly powder is dissolved in.

29. The method of claim 27, wherein the nanoparticle slurry is a suspension of the nanoparticle assembly within the second solvent.

30. The method of claim 27, wherein forming the hydrogen sensor further comprises submerging the temperature sensor within the nanoparticle slurry to coat the temperature sensor with the nanoparticle assembly.

31. The method of claim 3027, wherein the second solvent is evaporated from the nanoparticle slurry that coats the temperature sensor to deposit the nanoparticle assembly on the temperature sensor.

32-37. (canceled)
